(19) United States
(12) Patent Application Publication
(10) Pub. No.: US 2011/0262525 A1
(43) Pub. Date: Oct. 27, 2011

(54) METHODS OF TREATMENT

(75) Inventors: Yan Wang, Warren, NJ (US); Chen Zong, Metuchen, NJ (US); Cynthia Seidel-Dugan, Mountainside, NJ (US); Yaolin Wang, Short Hills, NJ (US); Siu-Long Yao, West Windsor, NJ (US); Brian Der-Hua Lu, Westfield, NJ (US); Mohamad H. Ladha, Parsippany, NJ (US)

(73) Assignee: SCHERING CORPORATION, KENILWORTH, NJ (US)

(21) Appl. No.: 12/518,409

(22) PCT Filed: Dec. 11, 2007
(86) PCT No.: PCT/US07/25398

§ 371 Date: Feb. 23, 2011

Related U.S. Application Data
(60) Provisional application No. 60/874,589, filed on Dec. 13, 2006.
(60) Provisional application No. 60/870,937, filed on Dec. 20, 2006.
(60) Provisional application No. 60/946,011, filed on Jun. 25, 2007.
(60) Provisional application No. 60/979,274, filed on Oct. 11, 2007.

(51) Int. Cl. A61K 39/395; A61K 31/4985; A61K 31/704; A61K 9/127; A61K 31/7072; A61K 31/56; A61K 31/501; A61K 38/46; A61K 39/07; A61K 38/00; A61K 38/18; A61K 38/21; A61K 38/20; A61P 35/00; A61P 35/02; A61K 31/519

(52) U.S. Cl. 424/450; 514/265.1; 514/210.21; 424/172.1; 514/250; 424/158.1; 514/34; 514/50; 514/171; 514/252.03; 424/94.6; 424/246.1; 514/19.2; 424/133.1; 514/7.7; 514/7.6; 424/85.7; 424/85.2

(57) ABSTRACT

This invention relates to compositions and methods useful for treating various cancers. Therapeutic combinations and methods of use thereof are also covered in the present application.

This application claims the benefit of U.S. provisional patent application No. 60/874,589, filed Dec. 13, 2006; U.S. provisional patent application No. 60/870,937, filed Dec. 20, 2006; U.S. provisional patent application No. 60/946,011, filed Jun. 25, 2007 and U.S. provisional patent application No. 60/979,274, filed Oct. 11, 2007; each of which is herein incorporated by reference in its entirety.

### FIELD OF THE INVENTION

The present invention relates to compositions and methods for treating or preventing cancer.

### BACKGROUND OF THE INVENTION

The insulin-like growth factors, also known as somatomedins, include insulin-like growth factor-I (IGF-I) and insulin-like growth factor-II (IGF-II) (Klapper, et al., (1983) Endocrinol. 112:2215 and Rinderknecht, et al., (1978) Febs. Lett. 89:283). These growth factors exert mitogenic activity on various cell types, including tumor cells (Macaulay, (1992) Br. J. Cancer 65:311), by binding to a common receptor named the insulin-like growth factor-1 receptor (IGF1R or IGFR1) (Sepp-Lorenzino, (1998) Breast Cancer Research and Treatment 47:235). Interaction of IGFs with IGF1R activates the receptor by triggering autophosphorylation of the receptor on tyrosine residues (Butler, et al., (1998) Comparative Biochemistry and Physiology 121:19). Once activated, IGF1R, in turn, phosphorylates intracellular targets to activate cellular signaling pathways. This receptor activation is critical for stimulation of tumor cell growth and survival. Therefore, inhibition of IGF1R activity represents a valuable potential method to treat or prevent growth of human cancers and other proliferative diseases.

Accordingly, therapies that inhibit IGF1R are useful for the treatment or prevention of certain cancers. Anti-IGF1R antibodies are useful therapies for treating or preventing the cancers. There are several anti-IGF1R antibodies that are known in the art (see e.g., WO 03/100008; WO 2002/53596; WO 04/71529; WO 03/106621; US2003/235582; WO 04/83248; WO 03/59951; WO 04/87756 or WO 2005/16970). Other small molecule IGF1R inhibitors are also known in the art.

Although there are IGF1R inhibitors known in the art that may be used to treat or prevent some cancers, there remains a need in the art for therapeutic compositions and methods for treating or preventing other cancers such as head and neck cancer, squamous cell carcinoma, solitary plasmacytoma, multiple myeloma and renal cell cancer.

### SUMMARY OF THE INVENTION

The present invention addresses this need, in part, by providing IGF1R inhibitors and combinations thereof that are effective at treating or preventing head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia (e.g., T-cell lineage, B-cell precursor lineage), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, marginal zone lymphoma, Burkitt Lymphoma, mycosis fungoides, seary syndrome, cutaneous T-cell lymphoma, peripheral T cell lymphoma, chronic myeloproliferative disorders, myelofibrosis, myeloid metaplasia, systemic mastocytosis), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors or liver cancer.

The present invention provides a method for treating or preventing a medical condition, in a subject, selected from the group consisting of head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia (e.g., T-cell lineage, B-cell precursor lineage), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors and liver cancer comprising administering a therapeutically effective amount of an one or more IGF1R inhibitors or pharmaceutical compositions thereof to the subject. In an embodiment of the invention, the IGF1R inhibitor is selected from the group consisting of

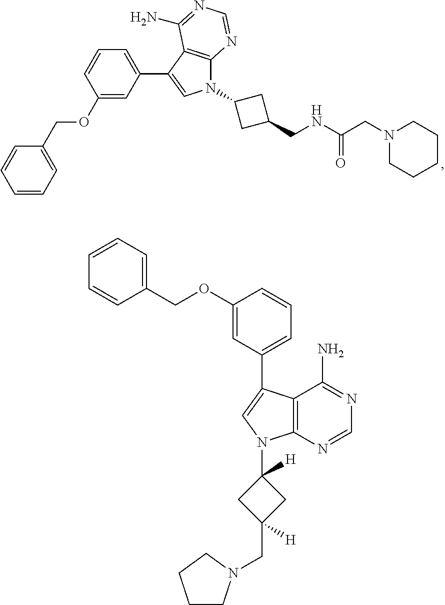

and an isolated antibody that binds specifically to IGF1R (e.g., human IGF1R) or an antigen-binding fragment thereof. In an embodiment of the invention, the antibody comprises: (a) a light chain variable region comprising amino acids 20-128 of SEQ ID NO: 2 and a heavy chain variable region comprising amino acids 20-137 of SEQ ID NO: 10 or 12; (b) a light chain variable region comprising amino acids 20-128 of SEQ ID NO: 4 and a heavy chain variable region comprising amino acids 20-137 of SEQ ID NO: 10 or 12; (c) a light chain variable region comprising amino acids 20-128 of SEQ ID NO: 6 and a heavy chain variable region comprising amino acids 20-137 of SEQ ID NO: 10 or 12; (d) a light chain variable region comprising amino acids 20-128 of SEQ ID NO: 8 and a heavy chain variable region comprising amino acids 20-137 of SEQ ID NO: 10 or 12; or any other IGF1R inhibitor set forth herein, for example, under the “IGF1R inhibitors” section below. In an embodiment of the invention, the IGF1R inhibitor is administered in association with one or more further anti-cancer chemotherapeutic agents or a pharmaceutical composition thereof. In an embodiment of the invention, the further anti-cancer chemotherapeutic agent is a member selected from the group consisting of teniposide

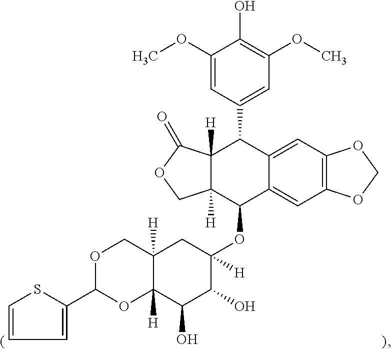

cisplatin

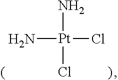

satraplatin, carboplatin

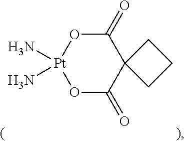

etoposide

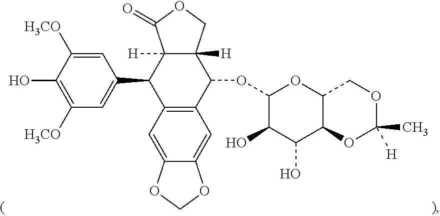

doxorubicin

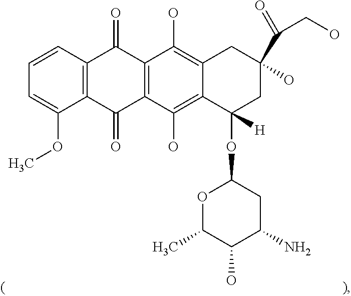

any liposomal formulation thereof such as Caelyx or Doxil®, cyclophosphamide

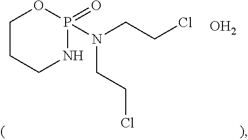

13-cis-retinoic acid

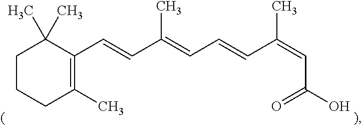

ifosfamide

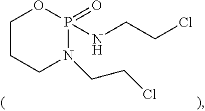

gemcitabine

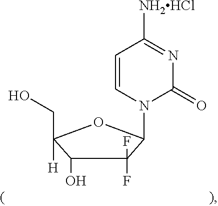

irinotecan

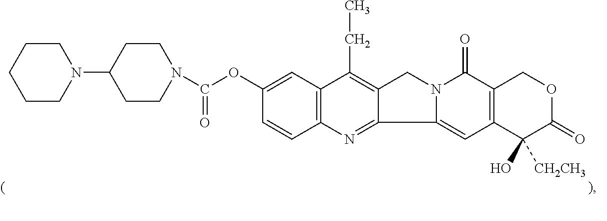

vincristine

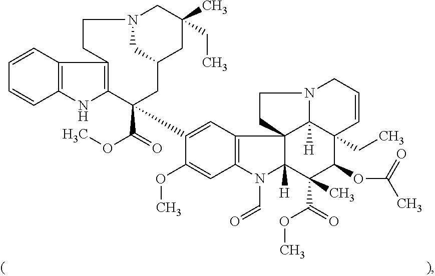

dactinomycin

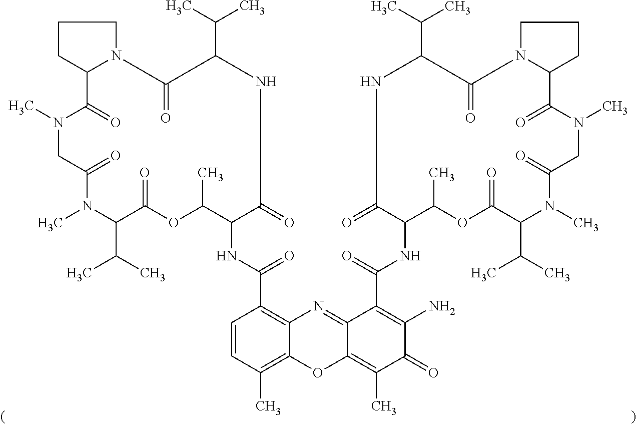

methotrexate

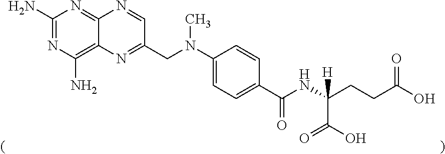

and any other chemotherapeutic agent set forth herein, for example, as set forth under the “Further Chemotherapeutics” section below. In an embodiment of the invention, the dosage of any anti-IGF1R antibody set forth herein is in the range of about 0.3-20 mg/kg of body weight or about 40-1200 mg/m². In an embodiment of the invention, the IGF1R inhibitor and the further anti-cancer therapeutic agent are administered simultaneously. In an embodiment of the invention, the IGF1R inhibitor and the further anti-cancer therapeutic agent are administered non-simultaneously. In an embodiment of the invention, the antibody comprises an IgG constant region. In an embodiment of the invention, the subject is a human (e.g., a child). In an embodiment of the invention, the IGF1R inhibitor is administered in association with an anti-cancer therapeutic procedure. In an embodiment of the invention, the anti-cancer therapeutic procedure is surgical tumorectomy and/or anti-cancer radiation treatment.

### BRIEF DESCRIPTION OF THE FIGURES

FIG. 1. IGF1R mRNA expression in primary head and neck tumor samples as measured using Taqman. Each point represents the normalized expression level of IGF1R in a single tissue sample. The stage of head and neck cancer in the patient from which each sample was obtained is marked I, II, III or IV. Values corresponding to normal tissue samples are marked “normal” and values corresponding to normal adjacent tissue, located adjacent to the tumor cells, but otherwise exhibiting normal characteristics, are marked “NAT”.

FIG. 2. Western blot analysis of the protein expression level of total IGF1R, IGF-I and IGF-II in various cell lines (top panel). Quantity of IGF1 and IGF2 secreted from various cell lines into the surrounding growth medium (ng IGF1 or IGF2 protein/10⁶ cells) (bottom panel).

FIGS. 3(a)-3(c). Inhibition of squamous cell carcinoma in vitro cell lines at various concentrations of anti-IGF1R antibody LCF/HCA. (a): cell line SCC 15; (b): cell line SCC 25; (c): cell line SCC 9.

FIG. 4. Expression level of IGF2 in primary stomach adenocarcinoma tumor tissue as compared to that of normal tissue.

FIG. 5. Expression levels of various protein in several gastic and ovarian cancer cell lines as measured by western blot analysis.

FIG. 6. In vitro growth inhibition of squamous cell carcinoma cancer cells (cell line SNU-16) at various concentrations of anti-IGF1R antibody LCF/HCA.

FIG. 7. Renal cell carcinoma tumor growth in a mouse xenograft model over time after exposure to anti-IGF1R antibody LCF/HCA or a control immunoglobulin.

FIG. 8. In vitro growth inhibition of melanoma cell line A375-SM when exposed to various concentrations of anti-IGF1R antibody LCF/HCA.

FIG. 9. In vivo growth inhibition of squamous cell carcinoma cell line SCC15 in mice when exposed to anti-IGF1R antibody LCF/HCA.

FIG. 10. Evaluation of tumor volume over time in EW5 (Ewins sarcoma), OS-1 (osteosarcoma) and OS-9 (osteosarcoma) xenograft tumor models.

### DETAILED DESCRIPTION OF THE INVENTION

The present invention comprises compositions and methods for treating or preventing non-cancerous medical disorders and cancer including head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia (e.g., T-cell lineage, B-cell precursor lineage), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors, liver cancer, gastric cancer, gigantism, pituitary adenoma, psoriasis and rhabdoid tumor of the kidney. The cancer may be treated or prevented by administering an IGF1R inhibitor, such as an anti-IGF1R antibody. The antibody can be associated with a further chemotherapeutic agent, such as an anti-cancer chemotherapeutic agent such as any of those set forth herein.

The terms IGF1R, IGFR1 and IGF-1R are synonymous and refer to insulin-like growth factor 1 receptor.

### IGF1R Inhibitors

The terms “IGF1R inhibitor” or “IGF1R antagonist” or the like include any substance that decreases the expression, ligand binding (e.g., binding to IGF-1 and/or IGF-2), kinase activity (e.g., autophosphorylation activity) or any other biological activity of IGF1R (e.g., mediation of anchorage independent cellular growth) and the phospho-IRS-1 level that will elicit a biological or medical response of a tissue, system, subject or patient that is being sought by the administrator (such as a researcher, doctor or veterinarian) which includes any measurable alleviation of the signs, symptoms and/or clinical indicia of both non-cancerous medical disorders and of cancer (e.g., tumor growth) and/or the prevention, slowing or halting of progression or metastasis of cancer (e.g., head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia (e.g., T-cell lineage, B-cell precursor lineage), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors, liver cancer, gastric cancer, gigantism, pituitary adenoma, psoriasis and rhabdoid tumor of the kidney to any degree.

In an embodiment of the invention, an IGF1R inhibitor that is administered to a patient in a method according to the invention is any isolated antibody or antigen-binding fragment thereof that binds specifically to insulin-like growth factor-1 receptor (e.g., human IGF1R) (e.g., monoclonal antibodies (e.g., fully human monoclonal antibodies), polyclonal antibodies, bispecific antibodies, Fab antibody fragments, F(ab)₂ antibody fragments, Fv antibody fragments (e.g., VH or VL), single chain Fv antibody fragments, dsFv antibody fragments, humanized antibodies, chimeric antibodies or anti-idiotypic antibodies) such as any of those disclosed in any of Burtrum et. al Cancer Research 63:8912-8921(2003); in French Patent Applications FR2834990, FR2834991 and FR2834900 and in PCT Application Publication Nos. WO 03/100008; WO 03/59951; WO 2006/13472; WO 04/71529; WO 03/106621; WO 04/83248; WO 04/87756, WO 05/16970; and WO 02/53596.

In an embodiment of the invention, an IGF1R inhibitor that is administered to a patient in a method according to the invention is an isolated anti-insulin-like growth factor-1 receptor (IGF1R) antibody comprising a mature 19D12/15H12 Light Chain-C, D, E or F (LCC, LCD, LCE or LCF) and a mature 19D12/15H12 heavy chain-A or B (HCA or HCB). In an embodiment of the invention, the antibody comprises the mature LCF and the mature HCA (LCF/HCA). In an embodiment of the invention, an IGF1R inhibitor that is administered to a patient in a method according to the invention is an isolated antibody that specifically binds to IGF1R that comprises one or more complementarity determining regions (CDRs) of 19D12/15H12 Light Chain-C, D, E or F and/or 19D12/15H12 heavy chain-A or B (e.g., all 3 light chain CDRs and all 3 heavy chain CDRs).

The amino acid and nucleotide sequences of the some antibody chains of the invention are shown below. Dotted, underscored type indicates the signal peptide. Solid underscored type indicates the CDRs. Plain type indicates the framework regions. Mature fragments lack the signal peptide.

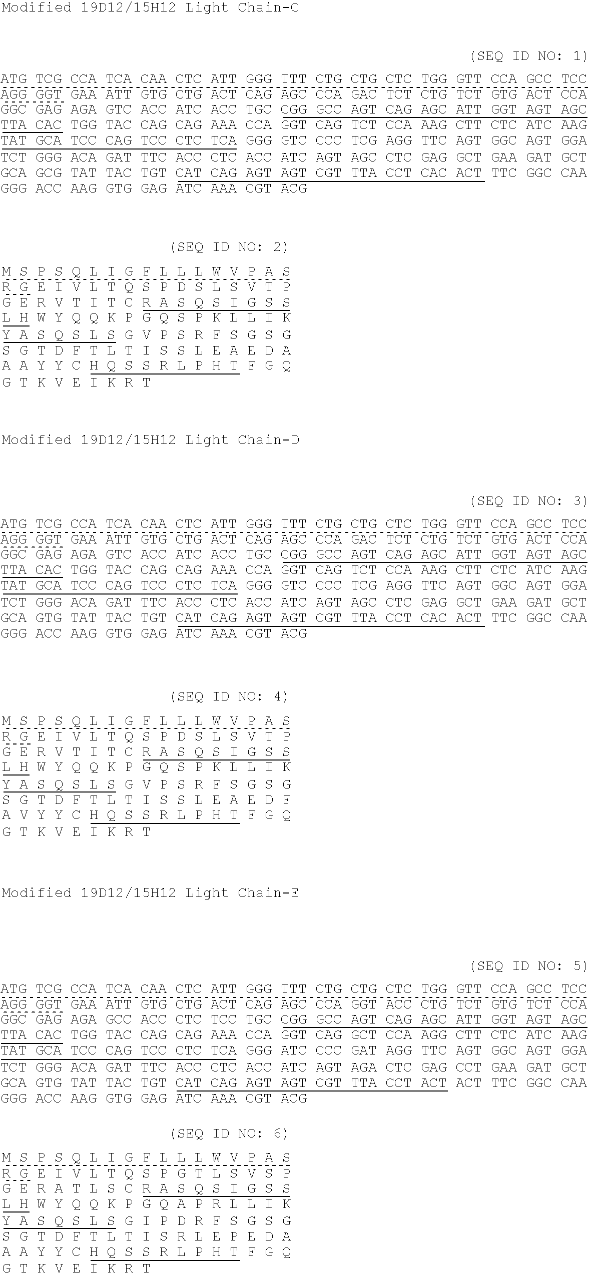

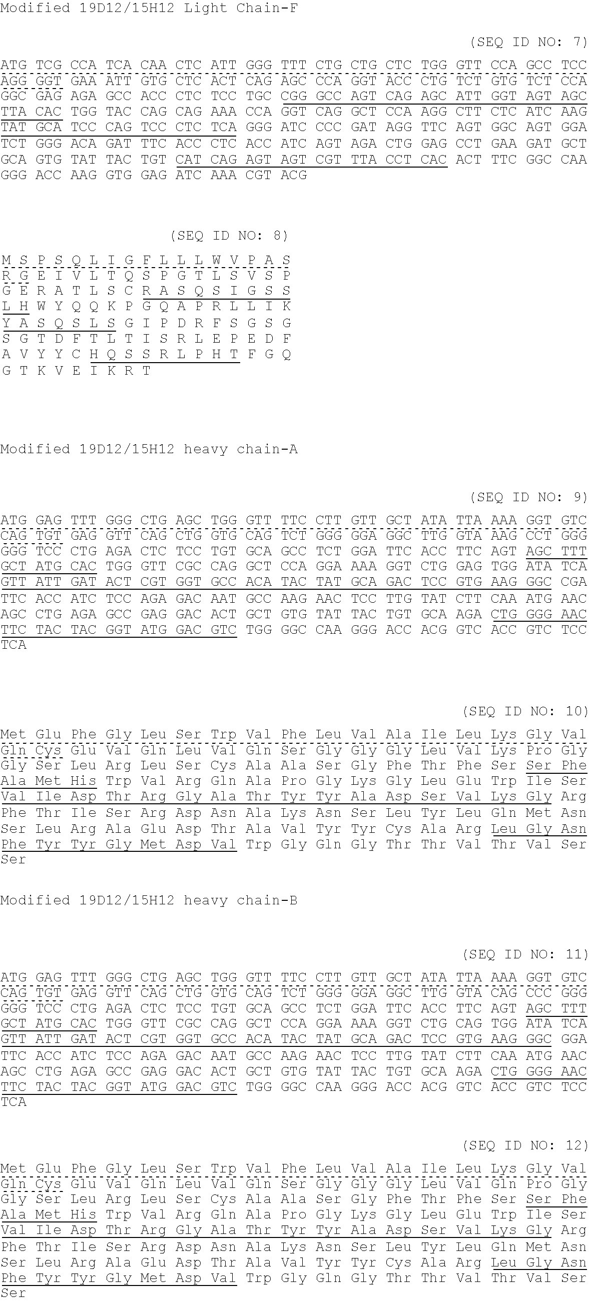

The present invention includes embodiments wherein the antibody chains set forth herein are substituted conservatively with one or more mutations that not significantly affect antibody binding activity.

Plasmids comprising a CMV promoter operably linked to the 15H12/19D12 light chains and heavy chains have been deposited at the American Type Culture Collection (ATCC); 10801 University Boulevard; Manassas, Va. 20110-2209 on May 21, 2003. The deposit name and the ATCC accession numbers for the plasmids are set forth below:
(i) CMV promoter-15H12/19D12 HCA (γ4)-

Deposit name: “15H12/19D12 HCA (γ4)”;

ATCC accession No.: PTA-5214;
(ii) CMV promoter-15H12/19D12 HCB (γ4)-

Deposit name: “15H12/19D12 HCB (γ4)”;

ATCC accession No.: PTA-5215;
(iii) CMV promoter-15H12/19D12 HCA (γ1)-

Deposit name: “15H12/19D12 HCA (γ1)”;

ATCC accession No.: PTA-5216;
(iv) CMV promoter-15H12/19D12 LCC (κ)-

Deposit name: “15H12/19D12 LCC (κ)”;

ATCC accession No.: PTA-5217;
(v) CMV promoter-15H12/19D12 LCD (κ)-

Deposit name: “15H12/19D12 LCD (κ)”;

ATCC accession No.: PTA-5218;
(vi) CMV promoter-15H12/19D12 LCE (κ)-

Deposit name: “15H12/19D12 LCE (κ)”;

ATCC accession No.: PTA-5219; and
(vii) CMV promoter-15H12/19D12 LCF (κ)-

Deposit name: “15H12/19D12 LCF (κ)”;

ATCC accession No.: PTA-5220;

The present invention includes methods and compositions (e.g., any disclosed herein) comprising anti-IGF1R antibodies and antigen-binding fragments thereof comprising any of the light and/or heavy immunoglobulin chains or mature fragments thereof located in any of the foregoing plasmids deposited at the ATCC.

In an embodiment of the invention, an antibody that binds “specifically” to human IGF1R binds with a Kd of about 10⁻⁸M or 10⁻⁷ M or a smaller number; or, in an embodiment of the invention, with a Kd of about 1.28×10⁻¹⁰ M or a smaller number by Biacore measurement or with a Kd of about 2.05×10⁻¹² M or a lower number by KinExA measurement. In another embodiment, an antibody that binds “specifically” to human IGF1R binds exclusively to human IGF1R and to no other protein in any specific amount.

In an embodiment of the invention, an IGF1R inhibitor that is administered to a patient in a method according to the invention comprises any light chain immunoglobulin and/or a heavy chain immunoglobulin as set forth in Published International Application No. WO 2002/53596 which is herein incorporated by reference in its entirety. For example, in an embodiment of the invention, the antibody comprises a light chain variable region comprising an amino acid sequence selected from the group consisting of SEQ ID NOs: 2, 6, 10, 14, 18, 22, 47 and 51 as set forth in WO 2002/53596 and/or a heavy chain variable region comprising an amino acid sequence selected from the group consisting of SEQ ID NOs: 4, 8, 12, 16, 20, 24, 45 and 49 as set forth in WO 2002/53596. In an embodiment of the invention, the antibody comprises a heavy and/or light chain selected from that of antibody 2.12.1; 2.13.2; 2.14.3; 3.1.1; 4.9.2; and 4.17.3 in WO 2002/53596.

In an embodiment of the invention, an IGF1R inhibitor that can be administered to a patient in a method according to the invention comprises any light chain immunoglobulin and/or a heavy chain immunoglobulin as set forth in Published International Application No. WO 2003/59951 which is herein incorporated by reference in its entirety. For example, in an embodiment of the invention, the antibody comprises a light chain variable region comprising an amino acid sequence selected from the group consisting of SEQ ID NOs: 54, 61 and 65 as set forth in WO 2003/59951 and/or a heavy chain variable region comprising an amino acids sequence selected from the group consisting of SEQ ID NOs: 69, 75, 79 and 83 as set forth in WO 2003/59951.

In an embodiment of the invention, an IGF1R inhibitor that can be administered to a patient in a method according to the invention comprises any light chain immunoglobulin and/or a heavy chain immunoglobulin as set forth in Published International Application No. WO 2004/83248 which is herein incorporated by reference in its entirety. For example, in an embodiment of the invention, the antibody comprises a light chain variable region comprising an amino acid sequence selected from the group consisting of SEQ ID NOs: 109, 111, 113, 115, 117, 119, 121, 123, 125, 127, 129, 131, 133, 135, 137, 139, 141 and 143 as set forth in WO 2004/83248 and/or a heavy chain variable region comprising an amino acids sequence selected from the group consisting of SEQ ID NOs: 108, 110, 112, 114, 116, 118, 120, 122, 124, 126, 128, 130, 132, 134, 136, 138, 140 and 142 as set forth in WO 2004/83248. In an embodiment of the invention, the antibody comprises a light and/or heavy chain selected from that of PINT-6A1; PINT-7A2; PINT-7A4; PINT-7A5; PINT-7A6; PINT-8A1; PINT-9A2; PINT-11A1; PINT-11A2; PINT-11A3; PINT-11A4; PINT-11A5; PINT-11A7; PINT-12A1; PINT-12A2; PINT-12A3; PINT-12A4 and PINT-12A5 in WO 2004/83248.

In an embodiment of the invention, an IGF1R inhibitor that can be administered to a patient in a method according to the invention comprises any light chain immunoglobulin and/or a heavy chain immunoglobulin as set forth in Published International Application No. WO 2003/106621 which is herein incorporated by reference in its entirety. For example, in an embodiment of the invention, the antibody comprises a light chain variable region comprising an amino acid sequence selected from the group consisting of SEQ ID NOs: 8-12, 58-69, 82-86, 90, 94, 96, 98, as set forth in WO 2003/106621 and/or a heavy chain variable region comprising an amino acids sequence selected from the group consisting of SEQ ID NOs: 7, 13, 70-81, 87, 88, 92 as set forth in WO 2003/106621.

In an embodiment of the invention, an IGF1R inhibitor that can be administered to a patient in a method according to the invention comprises any light chain immunoglobulin and/or a heavy chain immunoglobulin as set forth in Published International Application No. WO 2004/87756 which is herein incorporated by reference in its entirety. For example, in an embodiment of the invention, the antibody comprises a light chain variable region comprising an amino acid sequence of SEQ ID NO: 2 as set forth in WO 2004/87756 and/or a heavy chain variable region comprising an amino acid sequence of SEQ ID NO: 1 as set forth in WO 2004/87756.

In an embodiment of the invention, an IGF1R inhibitor that can be administered to a patient in a method according to the invention comprises any light chain immunoglobulin and/or a heavy chain immunoglobulin as set forth in Published International Application No. WO 2005/16970 which is herein incorporated by reference in its entirety. For example, in an embodiment of the invention, the antibody comprises a light chain variable region comprising an amino acid sequence of SEQ ID NO: 6 or 10 as set forth in WO 2005/16970 and/or a heavy chain variable region comprising an amino acid sequence of SEQ ID NO: 2 as set forth in WO 2005/16970.

In an embodiment of the invention, an anti-IGF1R antibody or antigen-binding fragment thereof of the invention comprises an immunoglobulin heavy chain variable region comprising an amino acid sequence selected from the group consisting of:

| (SEQ ID NO: 13) | |
|---|---|
| 1 | grlgqawrsl rlscaasgft fsdyymswir qapgkglewv syisssgstr |
| 51 | dyadsvkgrf tisrdnakns lylqmnslra edtavyycvr dgvettfyyy |
| 101 | yygmdvwgqg ttvtvssast kgpsvfplap csrstsesta algclvkdyf |
| 151 | pepvtvswns galtsgvhtf psca |
| (SEQ ID NO: 14) | |
| 1 | vqllesgggl vqpggslrls ctasgftfss yamnwvrqap gkglewvsai |
| 51 | sgsggttfya dsvkgrftis rdnsrttlyl qmnslraedt avyycakdlg |
| 101 | wsdsyyyyyg mdvwgqgttv tvss |
| (SEQ ID NO: 15) | |
| 1 | gpglvkpset lsltctvsgg sisnyywswi rqpagkglew igriytsgsp |
| 51 | nynpslksry tmsvdtsknq fslklnsvta adtavyycav tifgvviifd |
| 101 | ywgqgtivtv ss |
| (SEQ ID NO: 16) | |
| 1 | evqllesggg lvqpggslrl scaasgftfs syamswvrqa pgkglewvsa |
| 51 | isgsggityy adsvkgrfti srdnskntly lqmnslraed tavyycakdl |
| 101 | gygdfyyyyy gmdvwgqgtt vtvss |
| (SEQ ID NO: 17) | |
| 1 | pglvkpsetl sltctvsggs issyywswir qppgkglewi gyiyysgstn |
| 51 | ynpslksrvt isvdtsknqf slklssvtaa dtavyycart ysssfyyygm |
| 101 | dvwgqgttvt vss |
| (SEQ ID NO: 18) | |
| 1 | evqllesggg lvqpggslrl scaasgftfs syamswvrqa pgkglewvsg |
| 51 | itgsggstyy adsvkgrfti srdnskntly lqmnslraed tavyycakdp |
| 101 | gttvimswfd pwgqgtivtv ss |

In an embodiment of the invention, an anti-IGF1R antibody or antigen-binding fragment thereof of the invention comprises an immunoglobulin light chain variable region comprising an amino acid sequence selected from the group consisting of:

| (SEQ ID NO: 19) | |
|---|---|
| 1 | asvgdrvtft crasqdirrd lgwyqqkpgk apkrliyaas rlqsgvpsrf |
| 51 | sgsgsgteft ltisslqped fatyyclqhn nyprtfgqgt eveiirtvaa |
| 101 | psvfifppsd eqlksgtasv vcllnnfypr eakvqw |
| (SEQ ID NO: 20) | |
| 1 | diqmtqfpss lsasvgdrvt itcrasqgir ndlgwyqqkp gkapkrliya |
| 51 | asrlhrgvps rfsgsgsgte ftltisslqp edfatyyclq hnsypcsfgq |
| 101 | gtkleik |
| (SEQ ID NO: 21) | |
| 1 | sslsasvgdr vtftcrasqd irrdlgwyqq kpgkapkrli yaasrlqsgv |
| 51 | psrfsgsgsg teftltissl qpedfatyyc lqhnnyprtf gqgteveiir |
| (SEQ ID NO: 22) | |
| 1 | diqmtqspss lsasvgdrvt itcrasqgir sdlgwfqqkp gkapkrliya |
| 51 | asklhrgvps rfsgsgsgte ftltisrlqp edfatyyclq hnsypltfgg |
| 101 | gtkveik |
| (SEQ ID NO: 23) | |
| 1 | gdrvtitcra sqsistflnw yqqkpgkapk llihvasslq ggvpsrfsgs |
| 51 | gsgtdftlti sslqpedfat yycqqsynap ltfgggtkve ik |
| (SEQ ID NO: 24) | |
| 1 | ratlscrasq svrgrylawy qqkpgqaprl liygassrat gipdrfsgsg |
| 51 | sgtdftltis rlepedfavf ycqqygsspr tfgqgtkvei k |

In an embodiment of the invention, the anti-IGF1R antibody comprises a light chain immunoglobulin, or a mature fragment thereof (i.e., lacking signal sequence), or variable region thereof, comprising the amino acid sequence of:

| (SEQ ID NO: 25) | |
|---|---|
| 1 | mdmrvpaqll gllllwfpga rc [symbol] |
| 51 | [symbol] |
| 101 | [symbol] tvaapsvfif ppsdeqlksg |
| 151 | tasvvcllnn fypreakvqw kvdnalqsgn sqesvteqds kdstyslsst |
| 201 | ltlskadyek hkvyacevth qglsspvtks fnrgec; |
| (SEQ ID NO: 26) | |
| 1 | mdmrvpaqll gllllwfpga rc [symbol] |
| 51 | [symbol] |
| 101 | [symbol] tvaapsvfif ppsdeqlksg |
| 151 | tasvvcllnn fypreakvqw kvdnalqsgn sqesvteqds kdstyslsst |
| 201 | ltlskadyek hkvyacevth qglsspvtks fnrgec; |
| (SEQ ID NO: 27) | |
| 1 | mdmrvpaqll gllllwfpga rc [symbol] |
| 51 | [symbol] |
| 101 | [symbol] tvaapsvfif ppsdeqlksg |
| 151 | tasvvcllnn fypreakvqw kvdnalqsgn sqesvteqds kdstyslsst |
| 201 | ltlskadyek hkvyacevth qglsspvtks fnrgec; |
| | or |
| (SEQ ID NO: 28) | |
| 1 | mdmrvpaqll gllllwfpga rc [symbol] |
| 51 | [symbol] |
| 101 | [symbol] tvaapsvfif ppsdeqlksg |
| 151 | tasvvcllnn fypreakvqw kvdnalqsgn sqesvteqds kdstyslsst |
| 201 | ltlskadyek hkvyacevth qglssnytks fnrgec. |

In an embodiment of the invention, the signal sequence is amino acids 1-22 of SEQ ID NOs: 25-28. In an embodiment of the invention, the mature variable region is underscored. In an embodiment of the invention, the CDRs are in bold/italicized font. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a heavy chain immunoglobulin or a mature fragment thereof (i.e., lacking signal sequence), or a variable region thereof, comprising the amino acid sequence of:

| (SEQ ID NO: 29) | |
|---|---|
| 1 | mefglswvfl vaiikgvqc [symbol] |
| 51 | [symbol] |
| 101 | [symbol] ast |
| 151 | kgpsvfplap csrstsesta algclvkdyf pepvtvswns galtsgvhtf |
| 201 | pavlqssgly slssvvtvps snfgtqtytc nvdhkpsntk vdktverkcc |
| 251 | vecppcpapp vagpsvflfp pkpkdtlmis rtpevtcvvv dvshedpevq |
| 301 | fnwyvdgvev hnaktkpree qfnstfrvvs vltvvhqdwl ngkeykckvs |
| 351 | nkglpapiek tisktkgqpr epqvytlpps reemtknqvs ltclvkgfyp |
| 401 | sdiavewesn gqpennyktt ppmldsdgsf flyskltvdk srwqqgnvfs |
| 451 | csvmhealhn hytqkslsls pgk; |
| (SEQ ID NO: 30) | |
| 1 | mefglswvfl vaiikgvqc [symbol] |
| 51 | [symbol] |
| 101 | [symbol] astkgp |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwlngk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspgk; |
| (SEQ ID NO: 31) | |
| 1 | mefglswlfl vailkgvqc [symbol] |
| 51 | [symbol] |
| 101 | [symbol] astkgp |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwingk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspgk; |
| | or |
| (SEQ ID NO: 32) | |
| 1 | mefglswlfl vailkgvqc [symbol] |
| 51 | [symbol] |
| 101 | [symbol] |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwlngk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspqk. |

In an embodiment of the invention, the signal sequence is amino acids 1-19 of SEQ ID NOs: 29-32. In an embodiment of the invention, the mature variable region is underscored. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a light chain variable region comprising the amino acid sequence of any of SEQ ID NOs: 19-24 paired with a heavy chain variable region comprising an amino acid sequence of any of SEQ ID NOs: 13-18, respectively. In an embodiment of the invention, the anti-IGF1R antibody comprises a mature light chain variable region comprising an amino acid sequence of any of SEQ ID NOs: 25 or 26 paired with a heavy chain variable region comprising an amino acid sequence of any of SEQ ID NOs: 29 or 30. In an embodiment of the invention, the anti-IGF1R antibody comprises a mature light chain variable region comprising an amino acid sequence of any of SEQ ID NOs: 27 or 28 paired with a heavy chain variable region comprising an amino acid sequence of any of SEQ ID NOs: 31 or 32.

In an embodiment of the invention, an anti-IGF1R antibody or antigen-binding fragment thereof of the invention comprises an immunoglobulin heavy chain or mature fragment or variable region of 2.12.1 fx (SEQ ID NO: 33) (in an embodiment of the invention, the leader sequence is underscored; in an embodiment of the invention, the CDRs are in bold/italicized font):

| | |
|---|---|
| 1 | mefglswvfl vaiikgvqcq vqlvesgggl vkpggslrls caasg [symbol] |
| 51 | [symbol] wirqap gkglewvs [symbol] [symbol] [symbol] rftis rdnaknslyl |
| 101 | qmnslraedt avyycar [symbol] [symbol] [symbol] wgqgttv tvssastkgp |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwlngk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspgk |

In an embodiment of the invention, the anti-IGF1R antibody or antigen-binding fragment thereof of the invention comprises amino acids 20-470 of 2.12.1 fx (SEQ ID NO: 33).

In an embodiment of the invention, an anti-IGF1R antibody or antigen-binding fragment thereof of the invention comprises mature immunoglobulin heavy chain variable region 2.12.1 fx (amino acids 20-144 or SEQ ID NO: 33; SEQ ID NO: 34):

q vqlvesgggl vkpggslrls caasgftfsd yymswirqap
gkglewvsyi sssgstrdya dsvkgrftis rdnaknslyl
qmnslraedt avyycardgv ettfyyyyyg mdvwgqgttv tvss

In an embodiment of the invention, an anti-IGF1R antibody or antigen-binding fragment thereof of the invention comprises an immunoglobulin light chain or mature fragment or variable region 2.12.1 fx (SEQ ID NO: 35) (in an embodiment of the invention, the leader sequence is underscored; in an embodiment of the invention, the CDRs are in bold/italicized font):

| | |
|---|---|
| 1 | mdmrvpaqll qllllwfpga rcdiqmtqsp sslsasvgdr vtitc [symbol] |
| 51 | [symbol] yqq kpgkapkrli y [symbol] gv psrfsgsgsg teftltissl |
| 101 | qpedfatyyc [symbol] f gqgtkveikr tvaapsvfif ppsdeqlksg |
| 151 | tasvvcllnn fypreakvqw kvdnalqsgn sqesvteqds kdstyslsst |
| 201 | ltlskadyek hkvyacevth qglsspvtks fnrgec |

In an embodiment of the invention, an anti-IGF1R antibody or antigen-binding fragment thereof of the invention comprises amino acids 23-236 of 2.12.1 fx (SEQ ID NO: 35).

In an embodiment of the invention, an anti-IGF1R antibody or antigen-binding fragment thereof of the invention comprises mature immunoglobulin light chain variable region 2.12.1 fx (amino acids 23-130 of SEQ ID NO: 35; SEQ ID NO: 36):

diqmtqsp sslsasvgdr vtitcrasqd irrdlgwyqq
kpgkapkrli yaasrlqsgv psrfsgsgsg teftltissl
qpedfatyyc lqhnnyprtf gqgtkveikr

In an embodiment of the invention, an anti-IGF1R antibody or antigen-binding fragment thereof comprises or consists of a light chain immunoglobulin chain comprising or consisting of amino acids 23-236 of 2.12.1 fx (SEQ ID NO: 35) and a heavy chain immunoglobulin chain comprising or consisting of amino acids 20-470 of 2.12.1 fx (SEQ ID NO: 33).

In an embodiment of the invention, the anti-IGF1R antibody or antigen-binding fragment thereof comprises one or more 2.12.1 fx CDRs (e.g., 3 light chain CDRs and/or 3 heavy chain CDRs) as set forth above.

In an embodiment of the invention, an anti-IGF1R antibody or antigen-binding fragment thereof of the invention or antigen-binding fragment thereof comprises a humanized 7C10 immunoglobulin light chain variable region; version 1 (SEQ ID NO: 37):

| | |
|---|---|
| 1 | dvvmtqspls lpvtpgepas iscrssqsiv hsngntylqw ylqkpgqspq |
| 51 | lliykvsnrl ygvpdrfsgs gsgtdftlki srveaedvgv yycfqgshvp |
| 101 | wtfgqgtkve ik |

In an embodiment of the invention, an anti-IGF1R antibody or antigen-binding fragment thereof of the invention comprises humanized 7C10 immunoglobulin light chain variable region; version 2 (SEQ ID NO: 38):

| | |
|---|---|
| 1 | divmtqspls lpvtpgepas iscrssqsiv hsngntylqw ylqkpgqspq |
| 51 | lliykvsnrl ygvpdrfsgs gsgtdftlki srveaedvgv yycfqgshvp |
| 101 | wtfgqgtkve ik |

In an embodiment of the invention, an anti-IGF1R antibody or antigen-binding fragment thereof of the invention comprises a humanized 7C10 immunoglobulin heavy chain variable region; version 1 (SEQ ID NO: 39):

| | |
|---|---|
| 1 | qvqlqesgpg lvkpsetlsl tctvsgysit ggylwnwirq ppgkglewmg |
| 51 | yisydgtnny kpslkdriti srdtsknqfs lklssvtaad tavyycaryg |
| 101 | rvffdywgqg tlvtvss |

In an embodiment of the invention, an anti-IGF1R antibody or antigen-binding fragment thereof of the invention comprises the humanized 7C10 immunoglobulin heavy chain variable region; version 2 (SEQ ID NO: 40):

| | |
|---|---|
| 1 | qvqlqesgpg lvkpsetlsl tctvsgysit ggylwnwirq ppgkglewig |
| 51 | yisydgtnny kpslkdrvti srdtsknqfs lklssvtaad tavyycaryg |
| 101 | rvffdywgqg tlvtvss |

In an embodiment of the invention, an anti-IGF1R antibody or antigen-binding fragment thereof of the invention comprises the humanized 7C10 immunoglobulin heavy chain variable region; version 3 (SEQ ID NO: 41):

| | |
|---|---|
| 1 | qvqlqesgpg lvkpsetlsl tctvsgysis ggylwnwirq ppgkglewig |
| 51 | yisydgtnny kpslkdrvti svdtsknqfs lklssvtaad tavyycaryg |
| 101 | rvffdywgqg tivtvss |

In an embodiment of the invention, an anti-IGF1R antibody or antigen-binding fragment thereof of the invention comprises A12 immunoglobulin heavy chain variable region (SEQ ID NO: 42):

| | |
|---|---|
| 1 | evqlvqsgae vkkpgssvkv sckasggtfs syaiswvrqa pgqglewmgg |
| 51 | iipifgtany aqkfqgrvti tadkststay melsslrsed tavyycarap |
| 101 | lrflewstqd hyyyyymdvw gkgttvtvss |

In an embodiment of the invention, an anti-IGF1R antibody or antigen-binding fragment thereof of the invention comprises A12 immunoglobulin light chain variable region (SEQ ID NO: 43):

| | |
|---|---|
| 1 | sseltqdpav svalgqtvri tcqgdslrsy yaswyqqkpg qapvlviygk |
| 51 | nnrpsgipdr fsgsssgnta sltitgaqae deadyycnsr dnsdnrlifg |
| 101 | ggtkltvls |

### or (SEQ ID NO: 105):

| | |
|---|---|
| 1 | sseltqdpav svalgqtvri tcqgdslrsy yatwyqqkpg qapilviyge |
| 51 | nkrpsgipdr fsgsssgnta sltitgaqae deadyycksr dgsgqhlvfg |
| 101 | ggtkltvlg |

In an embodiment of the invention, an anti-IGF1R antibody or antigen-binding fragment thereof of the invention comprises 1A immunoglobulin heavy chain variable region (SEQ ID NO: 44):

| | |
|---|---|
| 1 | evqlvqsggg lvhpggslrl scagsgftfr nyamywvrqa pgkglewvsa |
| 51 | igsgggtyya dsvkgrftis rdnaknslyl qmnslraedm avyycarapn |
| 101 | wgsdafdiwg qgtmvtvss; |

optionally including one or more of the following mutations: R30, S30, N31, S31, Y94, H94, D104, E104.

In an embodiment of the invention, an anti-IGF1R antibody or antigen-binding fragment thereof of the invention comprises 1A immunoglobulin light chain variable region (SEQ ID NO: 45):

| | |
|---|---|
| 1 | diqmtqspss lsasvgdrvt itcrasqgis swlawyqqkp ekapksliya |
| 51 | asslqsgvps rfsgsgsgtd ftltisslqp edfatyycqq ynsypptfgp |
| 101 | gtkvdik |

;optionally including one or more of the following mutations: P96, I96, P100, Q100, R103, K103, V104, L104, D105, E105

In an embodiment of the invention, an anti-IGF1R antibody or antigen-binding fragment thereof of the invention comprises single chain antibody (fv) 8A1 (SEQ ID NO: 46):

| | |
|---|---|
| 1 | evqlvqsgae vkkpgeslti sckgpgynff nywigwvrqm pgkglewmgi |
| 51 | iyptdsdtry spsfqgqvti svdksistay lqwsslkasd tamyycarsi |
| 101 | rycpggrcys gyygmdvwgq gtmvtvssgg ggsggggsgg ggsseltqdp |
| 151 | avsvalgqtv ritcqgdslr syyaswyqqk pgqapvlviy gknnrpsgip |
| 201 | drfsgsssgn tasltitgaq aedeadyycn srdssgnhvv fgggtkltvl |
| 251 | g |

In an embodiment of the invention, an anti-IGF1R antibody or antigen-binding fragment thereof of the invention comprises single chain antibody (fv) 9A2 (SEQ ID NO: 47):

| | |
|---|---|
| 1 | qvqlvqsgae vrkpgasvkv scktsgytfr nydinwvrqa pgqglewmgr |
| 51 | isghygntdh aqkfqgrftm tkdtststay melrsltfdd tavyycarsq |
| 101 | wnvdywgrgt lvtvssgggg sggggsgggg salnfmltqp hsvsespgkt |
| 151 | vtisctrssg siasnyvqwy qqrpgssptt vifednrrps gvpdrfsgsi |
| 201 | dtssnsaslt isglktedea dyycqsfdst nlvvfgggtk vtvlg |

In an embodiment of the invention, an anti-IGF1R antibody or antigen-binding fragment thereof of the invention comprises single chain antibody (fv) 11A4 (SEQ ID NO: 48):

| | |
|---|---|
| 1 | evqllesggg lvqpggslrl scaasgftfs syamswvrqa pgkglewvsa |
| 51 | isgsggstyy adsvkgrfti srdnskntly lqmnslraed tavyycassp |
| 101 | yssrwysfdp wgqgtmvtvs sggggsgggg sggggsalsy eltqppsysv |
| 151 | spgqtatitc sgddlgnkyv swyqqkpgqs pvlviyqdtk rpsgiperfs |
| 201 | gsnsgniatl tisgtqavde adyycqvwdt gtvvfgggtk ltvlg |

In an embodiment of the invention, an anti-IGF1R antibody or antigen-binding fragment thereof of the invention comprises single chain antibody (fv) 7A4 (SEQ ID NO: 49):

| | |
|---|---|
| 1 | evqlvqsgae vkkpgeslti sckgsgynff nywigwvrqm pgkdlewmgi |
| 51 | iyptdsdtry spsfqgqvti svdksistay lqwsslkasd tamyycarsi |
| 101 | rycpggrcys gyygmdvwgq gtmvtvssgg gssggggsgg ggsseltqdp |
| 151 | avsvalgqtv ritcrgdslr nyyaswyqqk pgqapvlviy gknnrpsgip |
| 201 | drfsgsssgn tasltitgaq aedeadyycn srdssgnhmv fgggtkltvl |
| 251 | g |

In an embodiment of the invention, an anti-IGF1R antibody or antigen-binding fragment thereof of the invention comprises single chain antibody (fv) 11A1 (SEQ ID NO: 50):

| | |
|---|---|
| 1 | evqlvesggg vvqpgrslrl scaasgftfs dfamhwvrqi pgkglewlsg |
| 51 | lrhdgstayy agsvkgrfti srdnsrntvy lqmnslraed tatyycvtgs |
| 101 | gssgphafpv wgkgtlvtvs sggggsgggg sggggsalsy vltqppsasg |
| 151 | tpgqrvtisc sgsnsnigty tvnwfqqlpg tapklliysn nqrpsgvpdr |
| 201 | fsgsksgtsa slaisglqse deadyycaaw ddslngpvfg ggtkvtvlg |

In an embodiment of the invention, an anti-IGF1R antibody or antigen-binding fragment thereof of the invention comprises single chain antibody (fv) 7A6 (SEQ ID NO: 51)

| | |
|---|---|
| 1 | evqlvqsgae vkkpgeslti sckgsgynff nywigwvrqm pgkglewmgi |
| 51 | iyptdsdtry spsfqgqvti svdksistay lqwsslkasd tamyycarsi |
| 101 | rycpggrcys gyygmdvwgq gtlvtvssgg ggsggggsgg ggsseltqdp |
| 151 | avsvalgqtv ritcqgdslr syytnwfqqk pgqapllvvy aknkrpsgip |
| 201 | drfsgsssgn tasltitgaq aedeadyycn srdssgnhvv fgggtkltvl |
| 251 | g |

In an embodiment of the invention, an anti-IGF1R antibody or an antigen-binding fragment thereof (e.g., a heavy chain or light chain immunoglobulin) of the invention comprises one or more complementarity determining regions (CDR) selected from the group consisting of:

| | |
|---|---|
| sywmh; | (SEQ ID NO: 52) |
| einpsngrtnynekfkr; | (SEQ ID NO: 53) |
| grpdyygsskwyfdv; | (SEQ ID NO: 54) |
| rssqsivhsnvntyle; | (SEQ ID NO: 55) |
| kvsnrfs; | (SEQ ID NO: 56) |
| and | |
| fqgshvppt. | (SEQ ID NO: 57) |

In an embodiment of the invention, an anti-IGF1R antibody or an antigen-binding fragment thereof of the invention comprises a heavy chain immunoglobulin variable region selected from the group consisting of:

| (SEQ ID NO: 58) | |
|---|---|
| 1 | qvqlvqsgae vvkpgasvkl sckasgytft sywmhwvkqr pgqglewige |
| 51 | inpsngrtny nqkfqgkatl tvdkssstay mqlssltsed savyyfargr |
| 101 | pdyygsskwy fdvwgqgttv tvs; |
| (SEQ ID NO: 59) | |
| 1 | qvqfqqsgae lvkpgasvkl sckasgytft sylmhwikqr pgrglewigr |
| 51 | idpnnvvtkf nekfkskatl tvdkpsstay melssltsed savyycarya |
| 101 | ycrpmdywgq gttvtvss; |
| (SEQ ID NO: 60) | |
| 1 | qvqlqqsgae lvkpgasvkl sckasgytft sywmhwvkqr pgqglewige |
| 51 | inpsngrtny nekfkrkatl tvdkssstay mqlssltsed savyyfargr |
| 101 | pdyygsskwy fdvwgagttv tvs; |
| (SEQ ID NO: 61) | |
| 1 | qvqlqqsgae lmkpgasvki sckatgytfs sfwiewvkqr pghglewige |
| 51 | ilpgsggthy nekfkgkatf tadkssntay mqlssltsed savyycargh |
| 101 | syyfydgdyw gqgtsvtvss; |
| (SEQ ID NO: 62) | |
| 1 | qvqlqqpgsv lvrpgasvkl sckasgytft sswihwakqr pgqglewige |
| 51 | ihpnsgntny nekfkgkatl tvdtssstay vdlssltsed savyycarwr |
| 101 | ygspyyfdyw gqgttltvss; |
| (SEQ ID NO: 63) | |
| 1 | qvqlqqpgae lvkpgasvkl sckasgytft sywmhwvkqr pgrglewigr |
| 51 | idpnsggtky nekfkskatl tvdkpsstay mqlssltsed savyycaryd |
| 101 | yygssyfdyw gqgttltvss; |
| (SEQ ID NO: 64) | |
| 1 | qvqlvqsgae vvkpgasvkl sckasgytft sywmhwvkqr pgqglewige |
| 51 | inpsngrtny nqkfqgkatl tvdkssstay mqlssltsed savyyfargr |
| 101 | pdyygsskwy fdvwgqgttv tvs; |
| (SEQ ID NO: 65) | |
| 1 | qvqlqqsgae lvkpgasvkl sckasgytft sywmhwvkqr pgqglewige |
| 51 | inpsngrtny nekfkrkatl tvdkssstay mqlssltsed savyyfargr |
| 101 | pdyygsskwy fdvwgagttv tvss; |
| (SEQ ID NO: 66) | |
| 1 | qvqlvqsgae vvkpgasvkl sckasgytft sywmhwvkqr pgqglewige |
| 51 | inpsngrtny nqkfqgkatl tvdkssstay mqlssltsed savyyfargr |
| 101 | pdyygsskwy fdvwgqgttv tvss; |
| (SEQ ID NO: 67) | |
| 1 | qvqlqqsgae lvkpgasvkl sckasgytft sywmhwvkqr pgrglewigr |
| 51 | idpnsggtky nekfkskatl tvdkpsstay mqlssltsed savyycaryd |
| 101 | yygssyfdyw gqgttvtvss; |
| (SEQ ID NO: 68) | |
| 1 | qiqlqqsgpe lvrpgasvki sckasgytft dyyihwvkqr pgeglewigw |
| 51 | iypgsgntky nekfkgkatl tvdtssstay mqlssltsed savyfcargg |
| 101 | kfamdywgqg tsvtvss; |
| (SEQ ID NO: 69) | |
| 1 | qvqlqqsgae lvkpgasvkl sckasgytft sywmhwvkqr pgqglewige |
| 51 | inpsngrtny nekfkrkatl tvdkssstay mqlssltsed savyyfargr |
| 101 | pdyygsskwy fdvwgagttv tvss; |
| (SEQ ID NO: 70) | |
| 1 | qiqlqqsgpe lvkpgasvki sckasgytft dyyinwmkqk pgqglewigw |
| 51 | idpgsgntky nekfkgkatl tvdtssstay mqlssltsed tavyfcarek |
| 101 | ttyyyamdyw gqgtsvtvsa; |
| (SEQ ID NO: 71) | |
| 1 | vqlqqsgael mkpgasvkis ckasgytfsd ywiewvkqrp ghglewigei |
| 51 | lpgsgstnyh erfkgkatft adtssstaym qlnsltseds gvyyclhgny |
| 101 | dfdgwgqgtt ltvss; |
| | and |
| (SEQ ID NO: 72) | |
| 1 | qvqllesgae lmkpgasvki sckatgytfs sfwiewvkqr pghglewige |
| 51 | ilpgsggthy nekfkgkatf tadkssntay mqlssltsed savyycargh |
| 101 | syyfydgdyw gqgtsvtvss; |

and/or a light chain immunoglobulin variable region selected from the group consisting of:

| (SEQ ID NO: 73) | |
|---|---|
| 1 | dvlmtqipvs lpvslgdqas iscrssqiiv hnngntylew ylqkpgqspq |
| 51 | lliykvsnrf sgvpdrfsgs gsgtdftlki srveaedlgv yycfqgshvp |
| 101 | ftfgsgtkle ikr; |
| (SEQ ID NO: 74) | |
| 1 | dvlmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 75) | |
| 1 | dvlmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 76) | |
| 1 | dvlmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 77) | |
| 1 | dvlmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 78) | |
| 1 | dvlmtqtpls lpvslgdpas iscrssqxiv hsngntylew ylqkpgqspk |
| 51 | lliykvsnrf sgvpdrfsgs gsgtdftlki srveaedlgv yycfqgshvp |
| 101 | xtfgggtkle ikr; |
| (SEQ ID NO: 79) | |
| 1 | dvvmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 80) | |
| 1 | dvvmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 81) | |
| 1 | dvlmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 82) | |
| 1 | dvlmtqipvs lpvslgdqas iscrssqiiv hnngntylew ylqkpgqspq |
| 51 | lliykvsnrf sgvpdrfsgs gsgtdftlki srveaedlgv yycfqgshvp |
| 101 | ftfgsgtkle ikr; |
| (SEQ ID NO: 83) | |
| 1 | dvlmtqtpls lpvslgdqas iscrfsqsiv hsngntylew ylqksgqspk |
| 51 | lliykvsnrf sgvpdrfsgs gsgtdftlki srveaedlgv yycfqgshvp |
| 101 | rtfgggtkle ikr; |
| (SEQ ID NO: 84) | |
| 1 | dvlmtqtpls lpvslgdqas iscrssqsiv hsnvntylew ylqkpgqspk |
| 51 | lliykvsnrf sgvpdrfsgs gsgtdftlri srveaedlgi yycfqgshvp |
| 101 | ptfgggtkle ikr; |
| (SEQ ID NO: 85) | |
| 1 | dvvmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 86) | |
| 1 | elvmtqtpls lpvslgdgas iscrssqtiv hsngdtyldw flqkpgqspk |
| 51 | lliykvsnrf sgvpdrfsgs gsgtdftlki srveaedlgv yycfqgshvp |
| 101 | ptfgggtkle ikr; |
| (SEQ ID NO: 87) | |
| 1 | dvlmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 88) | |
| 1 | dvvmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 89) | |
| 1 | dvlmtqtpvs lsyslgdgas iscrssqsiv hstgntylew ylqkpgqspk |
| 51 | lliykisnrf sgvpdrfsgs gsgtdftlki srveaedlgv yycfclashap |
| 101 | rtfgggtkle ikr; |
| (SEQ ID NO: 90) | |
| 1 | dvlmtqtpls 1pvslgdqas isckssqsiv hssgntyfew ylqkpgqspk |
| 51 | lliykvsnrf sgvpdrfsgs gsgtdftlki srveaedlgv yycfqgship |
| 101 | ftfgsgtkle ikr; |
| (SEQ ID NO: 91) | |
| 1 | dieltqtpls lpvslgdqas iscrssqsiv hsngntylew ylqkpgqspk |
| 51 | lliykvsnrf sgvpdrfsgs gsgtdftlki srveaedlgv yycfqgshvp |
| 101 | ytfgggtkle ikr; |
| (SEQ ID NO: 92) | |
| 1 | dvlmtqtpls lpvslgdgas iscrssqsiv hsnvntylew ylqkpgqspk |
| 51 | lliykvsnrf sgvpdrfsgs gsgtdftlri srveaedlgi yycfqgshvp |
| 101 | ptfgggtkle ikr; |
| (SEQ ID NO: 93) | |
| 1 | dvvmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 94) | |
| 1 | dvlmtqtpls lpvslgdqas iscrssqsiv hsnvntylew ylqkpgqspk |
| 51 | lliykvsnrf sgvpdrfsgs gsgtdftlri srveaedlgi yycfqgshvp |
| 101 | ptfgggtkle ikr; |
| (SEQ ID NO: 95) | |
| 1 | dvvmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 96) | |
| 1 | dvlmtqtpls lpvslgdqas iscrsnqtil lsdgdtylew ylqkpgqspk |
| 51 | lliykvsnrf sgvpdrfsgs gsgtdftlki srveaedlgv yycfqgshvp |
| 101 | ptfgggtkle ikr; |
| (SEQ ID NO: 97) | |
| 1 | dvlmtqtpls lpvslgdqas iscrssqtiv hsngntylew ylqkpgqspk |
| 51 | lliykvtnrf sgvpdrfsgs gsgtdftlki srveaedlgv yycfqgthap |
| 101 | ytfgggtkle ikr; |
| | and |
| (SEQ ID NO: 98) | |
| 1 | dvlmtqtpls lpvslgdqas iscrssqsiv hsngntylew ylqkpgqspk |
| 51 | lliysissrf sgvpdrfsgs gsgtdftlki srvqaedlgv yycfqgshvp |
| 101 | ytfgggtkle ikr. |

The scope of the present invention includes methods wherein a patient is administered an anti-insulin-like growth factor receptor-1 (IGF1R) antibody wherein the variable region of the antibody is linked to any immunoglobulin constant region. In an embodiment of the invention, the light chain variable region is linked to a κ chain constant region. In an embodiment of the invention, the heavy chain variable region is linked to a γ1, γ2, γ3 or γ4 chain constant region. Any of the immunoglobulin variable regions set forth herein, in embodiments of the invention, can be linked to any of the foregoing constant regions.

Furthermore, the scope of the present invention comprises any antibody or antibody fragment comprising one or more CDRs (3 light chain CDRs and/or 3 heavy chain CDRs) and/or framework regions of any of the light chain immunoglobulin or heavy chain immunoglobulins set forth herein as identified by any of the methods set forth in Chothia et al., J. Mol. Biol. 186:651-663 (1985); Novotny and Haber, Proc. Natl. Acad. Sci. USA 82:4592-4596 (1985) or Kabat, E. A. et al., Sequences of Proteins of Immunological Interest, National Institutes of Health, Bethesda, Md., (1987)).

In an embodiment of the invention, the term “monoclonal antibody,” as used herein, refers to an antibody obtained from a population of substantially homogeneous antibodies, i.e., the individual antibodies comprising the population are identical except for possible naturally occurring mutations that may be present in minor amounts. As mentioned above, the monoclonal antibodies to be used in accordance with the present invention may be made by the hybridoma method first described by Kohler, et al., (1975) Nature 256: 495.

In an embodiment of the invention, a bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai, et al., (1990) Clin. Exp. Immunol. 79: 315-321, Kostelny, et al., (1992) J Immunol. 148:1547-1553. In addition, bispecific antibodies may be formed as “diabodies” (Holliger, et al., (1993) PNAS USA 90:6444-6448) or as “Janusins” (Traunecker, et al., (1991) EMBO J. 10:3655-3659 and Traunecker, et al., (1992) Int. J. Cancer Suppl. 7:51-52).

In an embodiment of the invention, the term “fully human antibody” refers to an antibody which comprises human immunoglobulin protein sequences only. A fully human antibody may contain non-human, e.g., murine, carbohydrate chains if produced in a mouse, in a mouse cell or in a hybridoma derived from a mouse cell. Similarly, “mouse antibody” refers to an antibody which comprises mouse immunoglobulin protein sequences only.

The present invention includes “chimeric antibodies”—an antibody which comprises a variable region of the present invention fused or chimerized with an antibody region (e.g., constant region) from another, human or non-human species (e.g., mouse, horse, rabbit, dog, cow, chicken). These antibodies may be used to modulate the expression or activity of IGF1R in the non-human species.

“Single-chain Fv” or “sFv” antibody fragments have the V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-IGF1R-specific single chain antibodies. For a review of sFv see Pluckthun in The Pharmacology of Monoclonal Antibodies, vol. 113, Rosenburg and Moore eds. Springer-Verlag, N.Y., pp. 269-315 (1994).

In an embodiment of the invention, “disulfide stabilized Fv fragments” and “dsFv” refer to immunoglobulins comprising a variable heavy chain (V_{H}) and a variable light chain (V_{L}) which are linked by a disulfide bridge.

Antigen-binding fragments of antibodies within the scope of the present invention also include F(ab)₂ fragments which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located.

An Fᵥ fragment is a V_{L} or V_{H} region.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. As discussed herein, any such antibody or antigen-binding fragment thereof is within the scope of the present invention.

The anti-IGF1R antibodies and fragments of the invention may also be conjugated to a chemical moiety. The chemical moiety may be, inter alia, a polymer, a radionuclide or a cytotoxic factor. In an embodiment of the invention, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antibody fragments may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibody molecules and fragments of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In an embodiment of the invention, an IGF1R inhibitor is BMS-577098

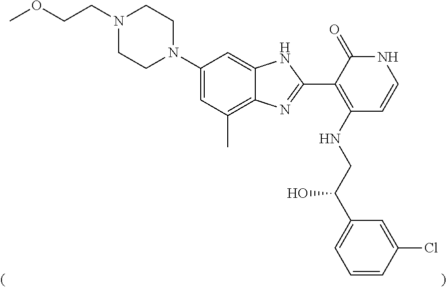

### or AEW-541

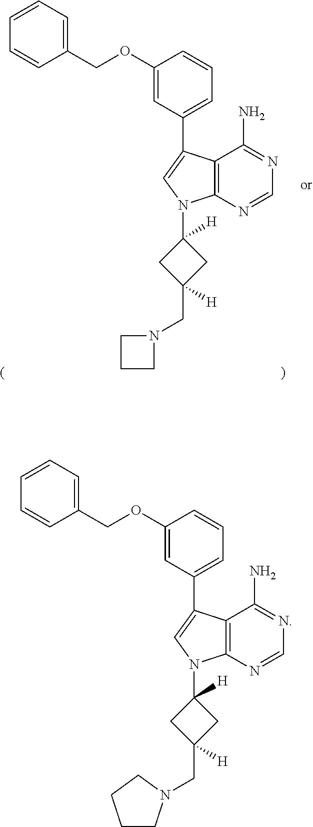

Methods of treating or preventing any medical condition set forth herein by administering these agents are within the scope of the present invention.

In an embodiment of the invention, an IGF1R inhibitor is any of the pyrimidine derivatives set forth in WO 03/48133, for example comprising the core structure:

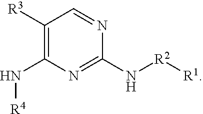

Methods of treating or preventing any medical disorder set forth herein by administering these agents are within the scope of the present invention.

In an embodiment of the invention, an IGF1R inhibitor is any of the tyrosine kinase inhibitors set forth in WO 03/35614, for example comprising the core structure:

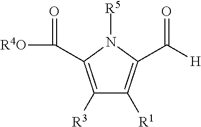

(e.g.,

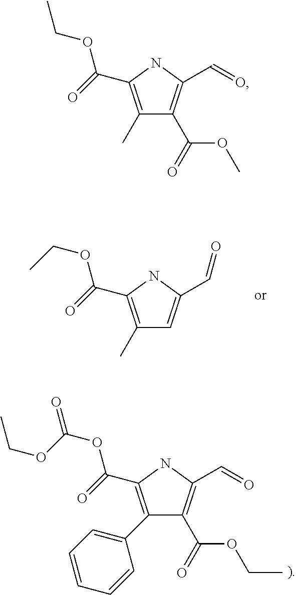

Methods of treating or preventing any medical disorder set forth herein by administering these agents are within the scope of the present invention.

In an embodiment of the invention, an IGF1R inhibitor is any of the tyrosine kinase inhibitors set forth in WO 03/35615, for example comprising the core structure:

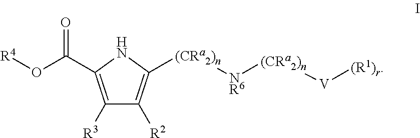

Methods of treating or preventing any medical disorder set forth herein by administering these agents are within the scope of the present invention.

In an embodiment of the invention, an IGF1R inhibitor is any of the tyrosine kinase inhibitors set forth in WO 03/35616, for example comprising the core structure:

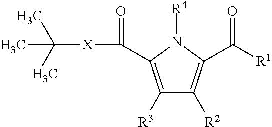

(e.g.,

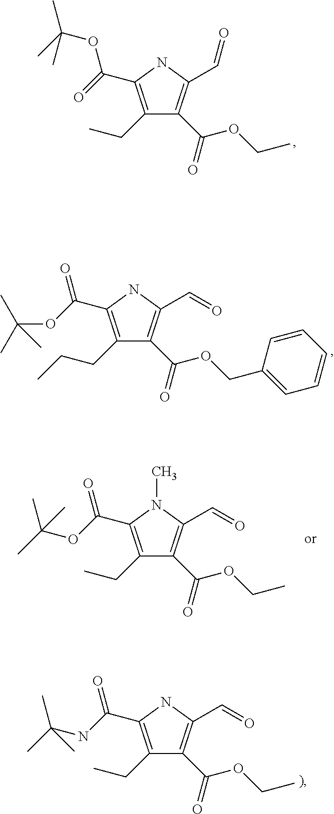

Methods of treating or preventing any medical disorder set forth herein by administering these agents are within the scope of the present invention.

In an embodiment of the invention, an IGF1R inhibitor is any of the tyrosine kinase inhibitors set forth in WO 03/35619, for example comprising the core structure:

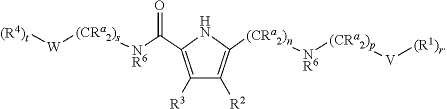

Methods of treating or any medical disorder set forth herein by administering these agents are within the scope of the present invention.

In an embodiment of the invention, an IGF1R inhibitor is a multitargeted kinase inhibitor which also inhibits e.g., VEGF-2R, Kit, FLT3 and/or PDGFR, for example, SU-11248 (e.g., sunitinib malate) or Bay43-9006 (sorafenib). Methods of treating or any medical disorder set forth herein by administering these agents is within the scope of the present invention.

In an embodiment of the invention, an IGF1R inhibitor is any of the compounds set forth in WO 03/24967, for example comprising the core structure:

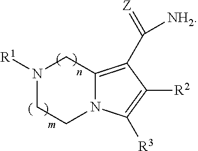

Methods of treating or preventing any medical disorder set forth herein by administering these agents are within the scope of the present invention.

In an embodiment of the invention, an IGF1R inhibitor is any of the compounds set forth in WO 04/30625, for example comprising the core structure:

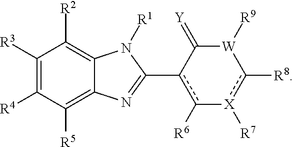

Methods of treating or preventing any medical disorder set forth herein by administering these agents are within the scope of the present invention.

In an embodiment of the invention, an IGF1R inhibitor is any of the compounds set forth in WO 04/30627, for example comprising the core structure:

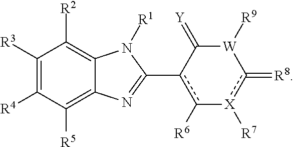

Methods of treating or preventing any medical disorder set forth herein by administering these agents are within the scope of the present invention.

In an embodiment of the invention, an IGF1R inhibitor is any of the heteroaryl-aryl ureas set forth in WO 00/35455, for example comprising the core structure:

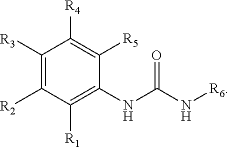

Methods of treating or preventing any medical disorder set forth herein by administering these agents are within the scope of the present invention.

In an embodiment of the invention, an IGF1R inhibitor is any of the peptides set forth in WO 03/27246. Methods of treating or preventing any medical disorder set forth herein by administering these agents are within the scope of the present invention.

In an embodiment of the invention, an IGF1R inhibitor is

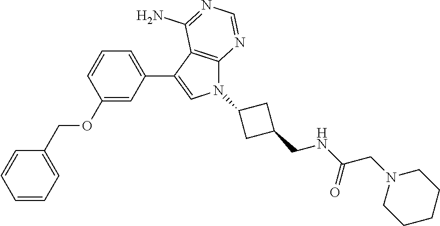

or any 4-amino-5-phenyl-7-cyclobutyl-pyrrolo[2,3-d]pyrimidine derivative disclosed in PCT Application Publication No. WO 02/92599. Methods of treating or preventing any medical disorder set forth herein by administering these agents are within the scope of the present invention.

### Generation of Antibodies

Any suitable method can be used to elicit an antibody with the desired biologic properties to inhibit IGF1R. It is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Other suitable techniques involve selection of libraries of antibodies in phage or similar vectors. See, e.g., Huse et al., Science 246:1275-1281 (1989); and Ward et al., Nature 341:544-546 (1989). The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. As discussed above, suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. Further methods for producing chimeric, humanized and human antibodies are well known in the art. See, e.g., U.S. Pat. No. 5,530,101, issued to Queen et al, U.S. Pat. No. 5,225,539, issued to Winter et al, U.S. Pat. No. 4,816,397 issued to Boss at al, all of which are incorporated by reference in their entirety.

Mammalian cell lines available as hosts for expression of antibodies of the invention are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion thereof, the light chain and/or antigen-binding portion thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

A convenient plasmid system useful for producing an anti-IGF1R antibody or antigen-binding fragment thereof is set forth in published U.S. application no. US2005/0176099 (see also WO2005/47512).

### Further Chemotherapeutics

The present invention provides methods for treating or preventing any of the medical disorders set forth herein by administering a therapeutically acceptable dosage or amount of an anti-IGF1R antibody or antigen-binding fragment thereof of the invention, e.g., as set forth herein, in association with a further chemotherapeutic agent (e.g., an anti-cancer chemotherapeutic agent, and/or an anti-emetic chemotherapeutic agent). For example, further chemotherapeutic agents include erlotinib, dasatanib, nilotinib, decatanib, panitumumab, amrubicin, oregovomab, Lep-etu (“liposome entrapped paclitaxel-Easy to Use”), nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763 or AT-9263.

Abraxane is an injectable suspension of paclitaxel protein-bound particles comprising an albumin-bound form of paclitaxel with a mean particle size of approximately 130 nanometers. Abraxane is supplied as a white to yellow, sterile, lyophilized powder for reconstitution with 20 mL of 0.9% Sodium Chloride Injection, USP prior to intravenous infusion. Each single-use vial contains 100 mg of paclitaxel and approximately 900 mg of human albumin. Each milliliter (mL) of reconstituted suspension contains 5 mg paclitaxel. Abraxane is free of solvents and is free of cremophor (polyoxyethylated castor oil).

In an embodiment of the invention, an antibody or antigen-binding fragment thereof of the invention is provided in association with romidepsin (FK-228;

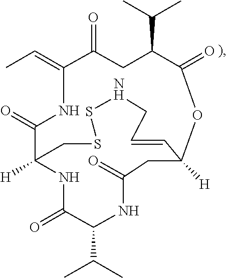

### ADS-100380,

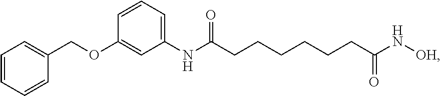

### CG-781

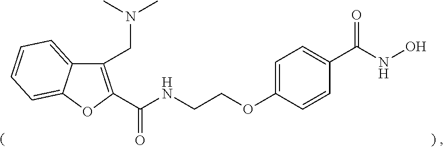

### CG-1521

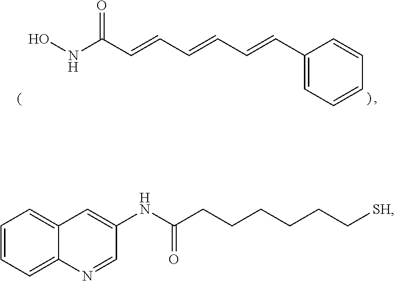

### SB-556629

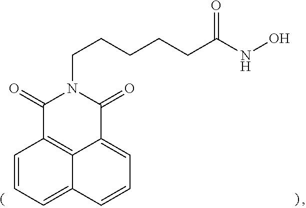

chlamydocin

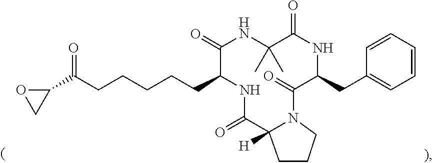

### JNJ-16241199

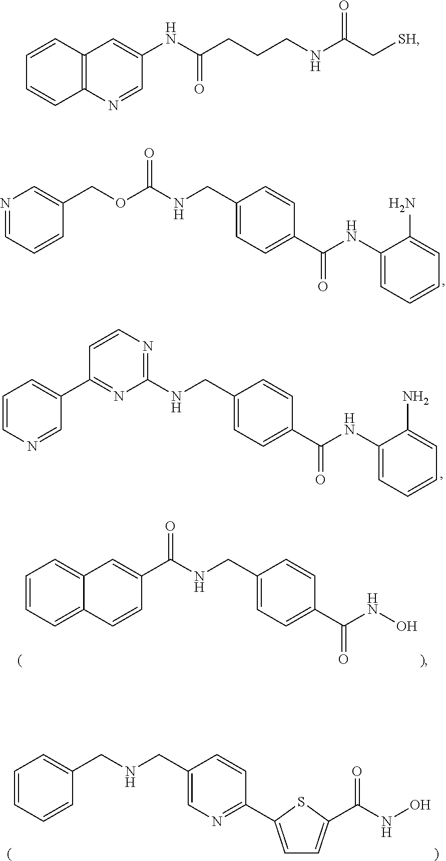

or vorinostat (SAHA;

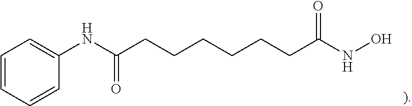

In an embodiment of the invention, an antibody or antigen-binding fragment thereof of the invention is provided in association with etoposide (VP-16;

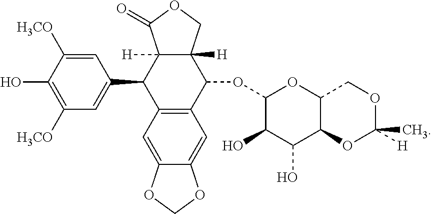

In an embodiment of the invention, an antibody or antigen-binding fragment thereof of the invention is provided in association with gemcitabine

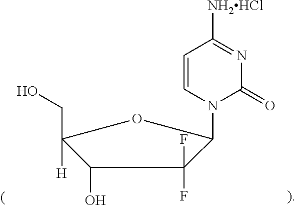

In an embodiment of the invention, an antibody or antigen-binding fragment thereof of the invention is provided in association with any compound disclosed in published U.S. patent application no. U.S. 2004/0209878A1 (e.g., comprising a core structure represented by

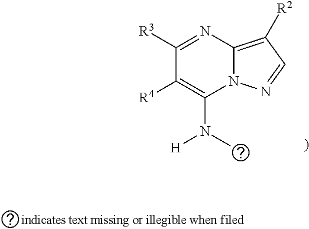

or doxorubicin

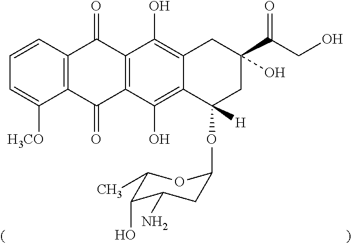

including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, an antibody or antigen-binding fragment thereof of the invention is provided in association with 5′-deoxy-5-fluorouridine

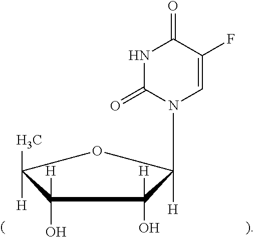

In an embodiment of the invention, an antibody or antigen-binding fragment thereof of the invention is provided in association with vincristine

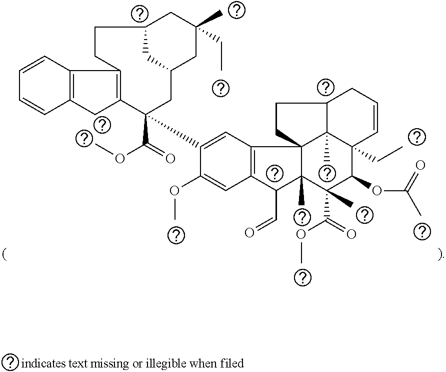

In an embodiment of the invention, an antibody or antigen-binding fragment thereof of the invention is provided in association with temozolomide

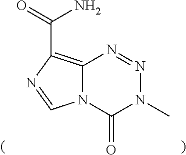

any CDK inhibitor such as ZK-304709, Seliciclib (R-roscovitine)

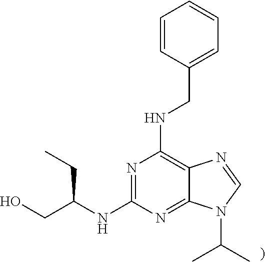

;any MEK inhibitor such as PD0325901

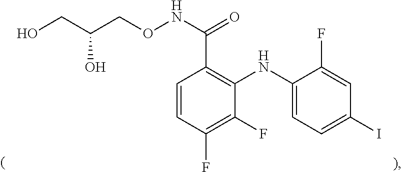

AZD-6244; capecitabine (5′-deoxy-5-fluoro-N-[(pentyloxy)carbonyl]-cytidine); or L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate

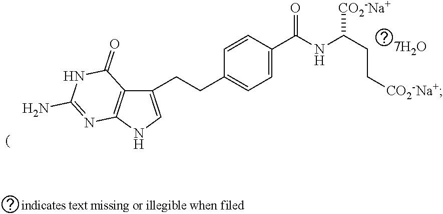

Pemetrexed disodium heptahydrate).

In an embodiment of the invention, an antibody or antigen-binding fragment thereof of the invention is provided in association with camptothecin

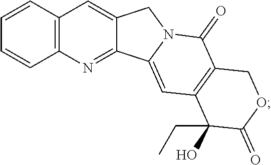

Stork et al., J. Am. Chem. Soc. 93(16): 4074-4075 (1971); Beisler et al., J. Med. Chem. 14(11): 1116-1117 (1962)), irinotecan

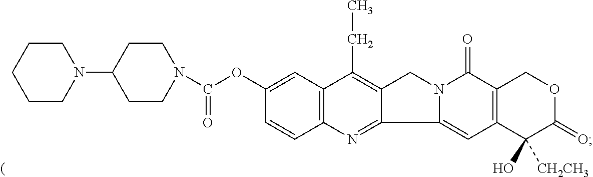

sold as Camptosar®; Pharmacia & Upjohn Co.; Kalamazoo, Mich.); a combination of irinotecan, 5-fluorouracil and leucovorin; or PEG-labeled irinotecan.

In an embodiment of the invention, an antibody or antigen-binding fragment thereof of the invention is provided in association with the FOLFOX regimen (oxaliplatin

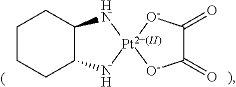

together with infusional fluorouracil

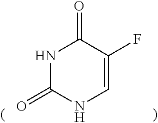

and folinic acid

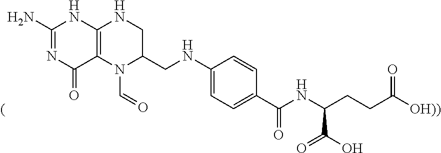

(Chaouche et al., Am. J. Clin. Oncol. 23(3):288-289 (2000); de Gramont et al., J. Clin. Oncol. 18(16):2938-2947 (2000)).

In an embodiment of the invention, an antibody or antigen-binding fragment thereof of the invention is provided in association with an antiestrogen such as

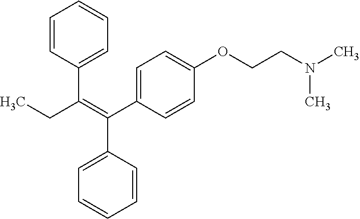

(tamoxifen; sold as Nolvadex® by AstraZeneca Pharmaceuticals LP; Wilmington, Del.) or

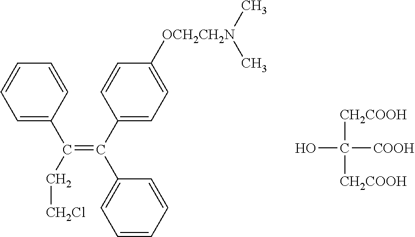

(toremifene citrate; sold as Fareston® by Shire US, Inc.; Florence, Ky.).

In an embodiment of the invention, an antibody or antigen-binding fragment thereof of the invention is provided in association with an aromatase inhibitor such as

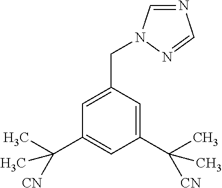

(anastrazole; sold as Arimidex® by AstraZeneca Pharmaceuticals LP; Wilmington, Del.),

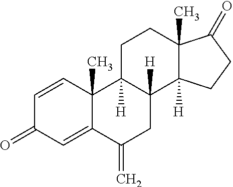

(exemestane; sold as Aromasin® by Pharmacia Corporation; Kalamazoo, Mich.) or

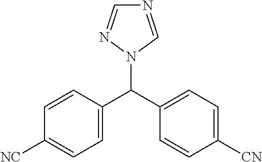

(letrozole; sold as Femara® by Novartis Pharmaceuticals Corporation; East Hanover, N.J.).

In an embodiment of the invention, an antibody or antigen-binding fragment thereof of the invention is provided in association with an estrogen such as DES(diethylstilbestrol),

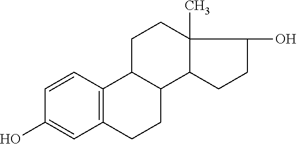

(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, an antibody or antigen-binding fragment thereof of the invention is provided in association with anti-angiogenesis agents including bevacizumab (Avastin™; Genentech; San Francisco, Calif.), the anti-VEGFR-2 antibody IMC-1C11, other VEGFR inhibitors such as: CHIR-258

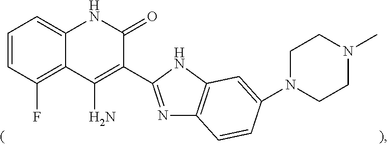

any of the inhibitors set forth in WO2004/13145 (e.g., comprising the core structural formula:

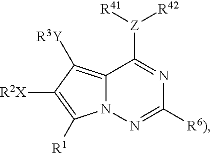

WO2004/09542 (e.g.,comprising the core structural formula:

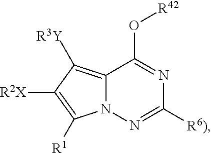

WO00/71129 (e.g., comprising the core structural formula:

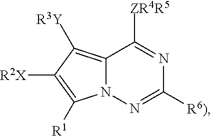

WO2004/09601 (e.g., comprising the core structural formula:

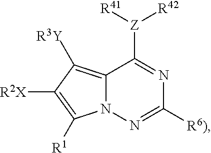

WO2004/01059 (e.g., comprising the core structural formula:

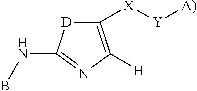

WO01/29025 (e.g., comprising the core structural formula:

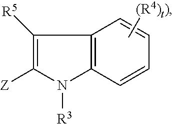

WO02/32861 (e.g., comprising the core structural formula:

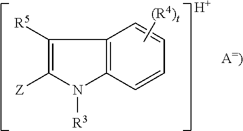

or set forth in WO03/88900 (e.g., comprising the core structural formula

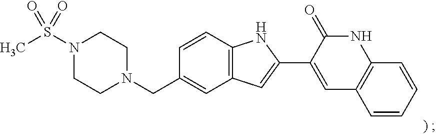

3-[5-(methylsulfonylpiperadinemethyl)-indolyl]-quinolone; Vatalanib

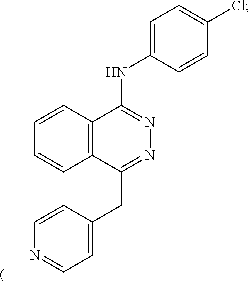

### PTK/ZK; CPG-79787; ZK-222584), AG-013736

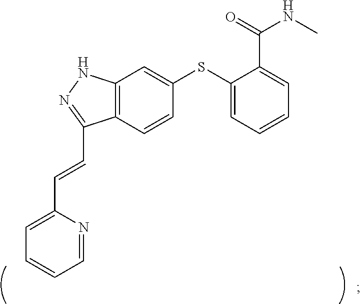

and the VEGF trap (AVE-0005), a soluble decoy receptor comprising portions of VEGF receptors 1 and 2.

In an embodiment of the invention, an antibody or antigen-binding fragment thereof of the invention is provided in association with a LHRH (Lutenizing hormone-releasing hormone) agonist such as the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4];

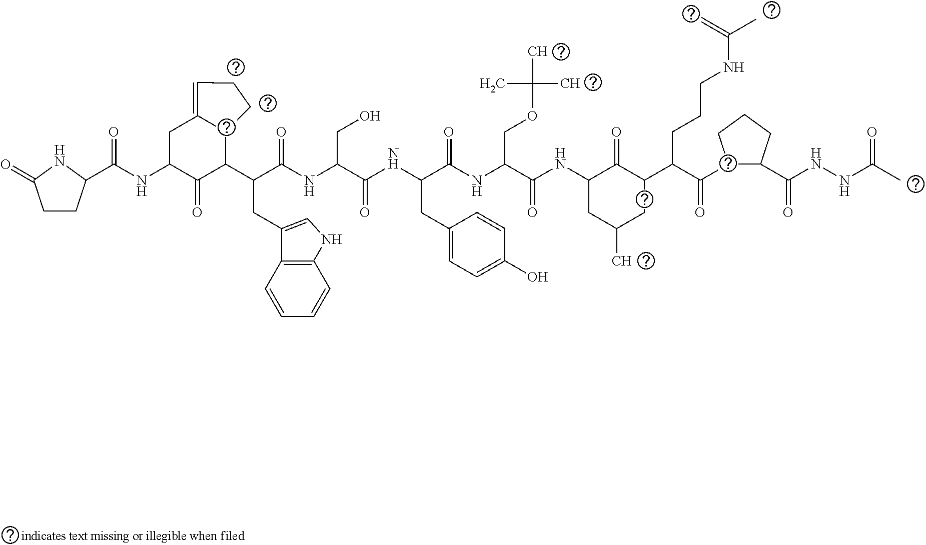

(goserelin acetate; sold as Zoladex® by AstraZeneca UK Limited; Macclesfield, England),

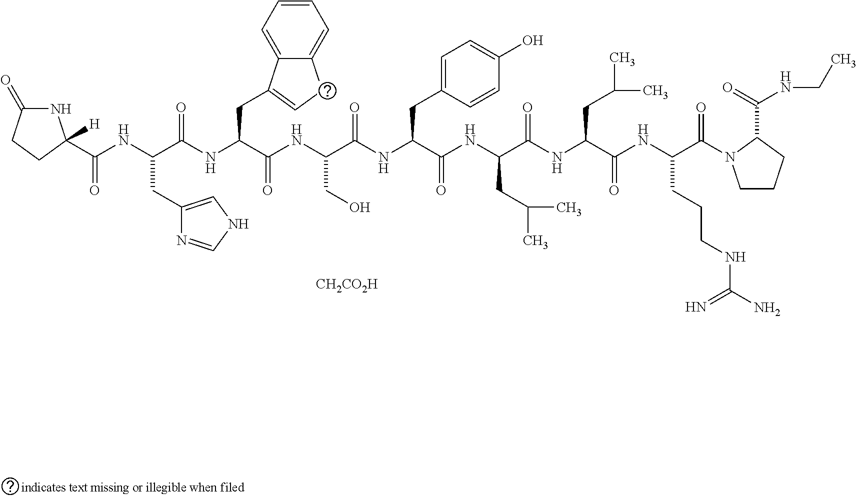

(leuprolide acetate; sold as Eligard® by Sanofi-Synthelabo Inc.; New York, N.Y.) or

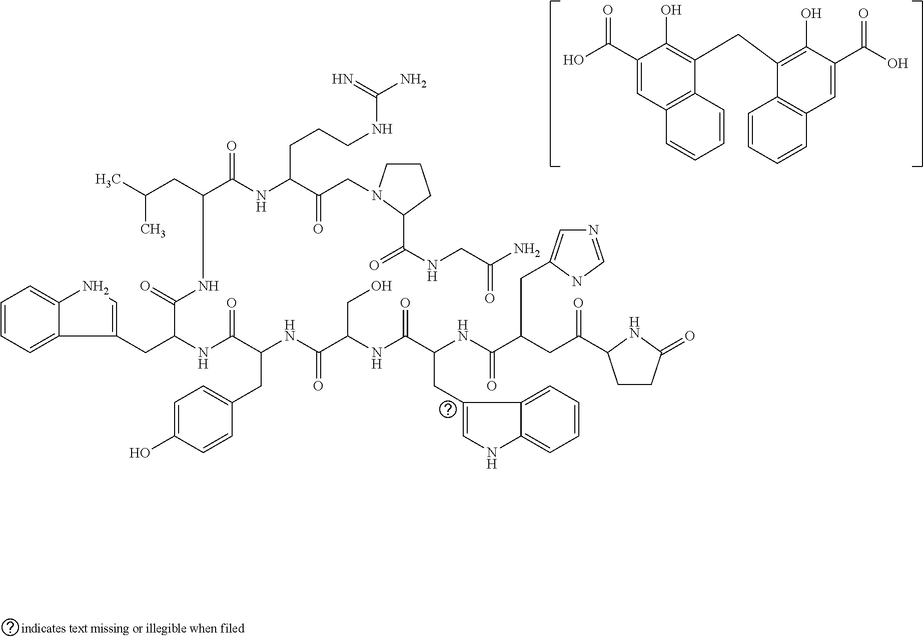

(triptorelin pamoate; sold as Trelstar® by Pharmacia Company, Kalamazoo, Mich.).

In an embodiment of the invention, an antibody or antigen-binding fragment thereof of the invention is provided in association with sunitinib or sunitinib malate

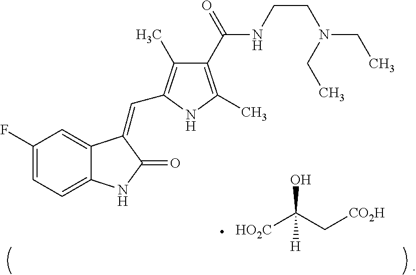

In an embodiment of the invention, an antibody or antigen-binding fragment thereof of the invention is provided in association with a progestational agent such as

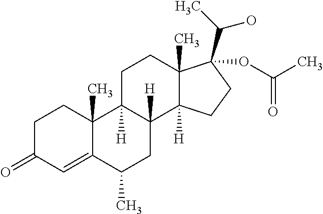

(medroxyprogesterone acetate; sold as Provera® by Pharmacia & Upjohn Co.; Kalamazoo, Mich.),

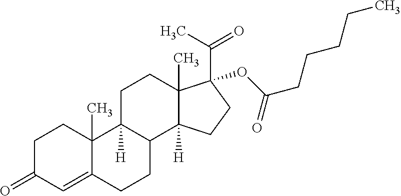

(hydroxyprogesterone caproate; 17-((1-Oxohexyl)oxy)pregn-4-ene-3,20-dione;), megestrol acetate or progestins.

In an embodiment of the invention, an antibody or antigen-binding fragment thereof of the invention is provided in association with selective estrogen receptor modulator (SERM) such as

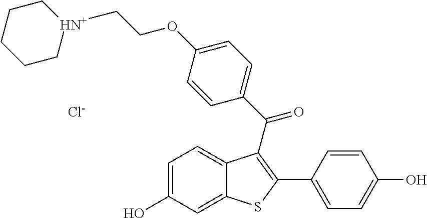

(raloxifene; sold as Evista® by Eli Lilly and Company; Indianapolis, Ind.).

In an embodiment of the invention, an antibody or antigen-binding fragment thereof of the invention is provided in association with an anti-androgen including, but not limited to:

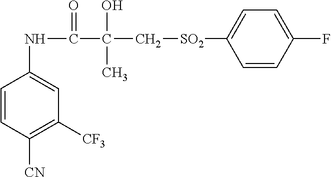

(bicalutamide; sold at CASODEX® by AstraZeneca Pharmaceuticals LP; Wilmington, Del.);

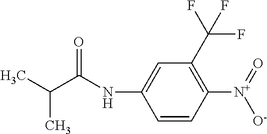

(flutamide; 2-methyl-N-[4-nitro-3(trifluoromethyl)phenyl]propanamide; sold as Eulexin® by Schering Corporation; Kenilworth, N.J.);

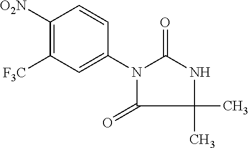

(nilutamide; sold as Nilandron® by Aventis Pharmaceuticals Inc.; Kansas City, Mo.) and

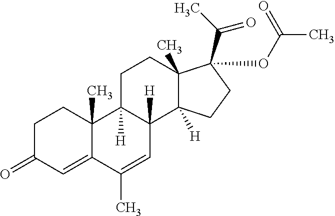

(Megestrol acetate; sold as Megace® by Bristol-Myers Squibb).

In an embodiment of the invention, an antibody or antigen-binding fragment thereof of the invention is provided in association with one or more inhibitors which antagonize the action of the EGF Receptor or HER2, including, but not limited to, CP-724714

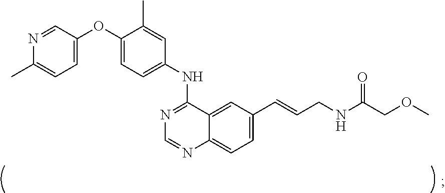

### TAK-165

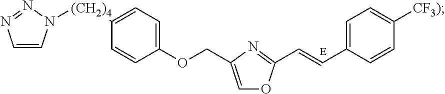

### HKI-272

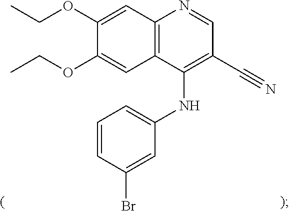

### OSI-774

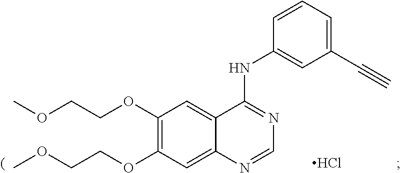

erlotinib, Hidalgo et al., J. Clin. Oncol. 19(13): 3267-3279 (2001)), Lapatanib

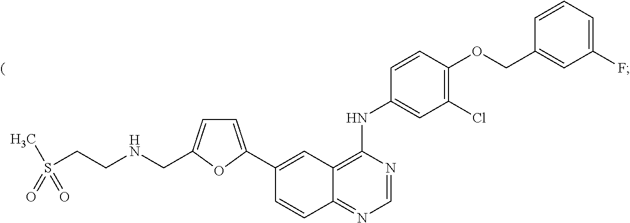

GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (CI-1033;

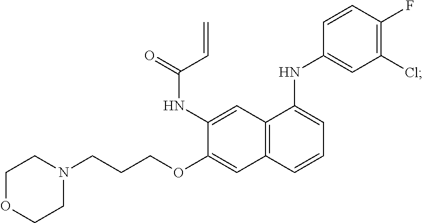

Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit Rev Oncol Hematol. 38(1):17-23 (2001)), erbitux (U.S. Pat. No. 6,217,866; IMC-C225, cetuximab; Imclone; New York, N.Y.), EKB-569

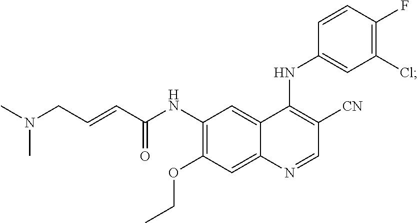

Wissner et al., J. Med. Chem. 46(1): 49-63 (2003)), PKI-166

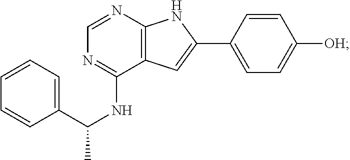

CGP-75166), GW-572016, any anti-EGFR antibody and any anti-HER2 antibody.

In an embodiment of the invention, an antibody or antigen-binding fragment thereof of the invention is provided in association with:

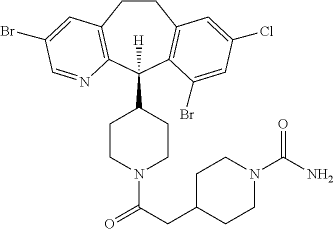

(Ionafarnib; Sarasar™; Schering-Plough; Kenilworth, N.J.). In another embodiment, one of the following FPT inhibitors is provided in association with an antibody or antigen-binding fragment thereof of the invention:

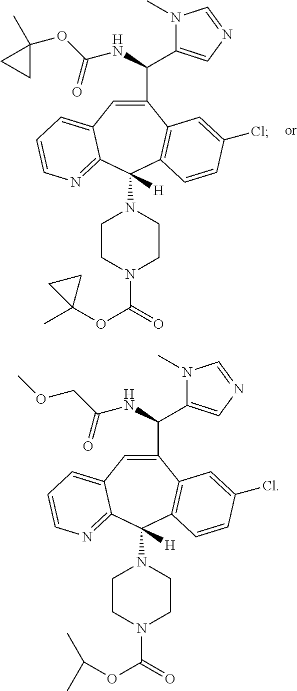

Other FPT inhibitors, that can be provided in association with an antibody or antigen-binding fragment thereof of the invention, include BMS-214662

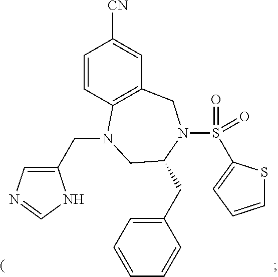

Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone];

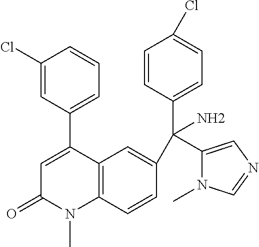

sold as Zarnestra™; Johnson & Johnson; New Brunswick, N.J.).

In an embodiment of the invention, an antibody or antigen-binding fragment thereof of the invention is provided in association with

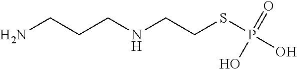

### (Amifostine);

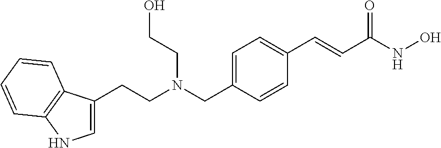

### (NVP-LAQ824; Atadja et al., Cancer Research 64: 689-695 (2004)),

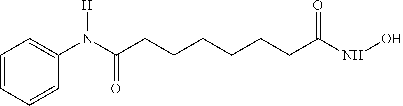

(suberoyl analide hydroxamic acid),

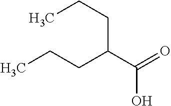

(Valproic acid; Michaelis et al., Mol. Pharmacol. 65:520-527 (2004)),

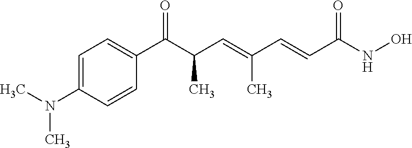

(trichostatin A),

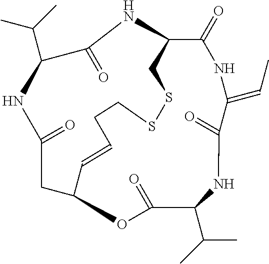

### (FK-228; Furumai et al., Cancer Research 62: 4916-4921 (2002)),

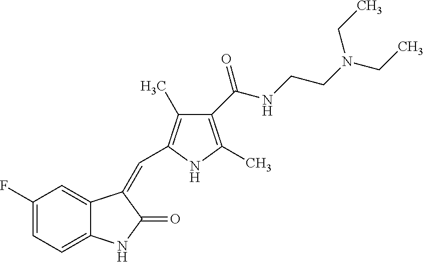

(SU11248; Mendel et al., Clin. Cancer Res. 9(1):327-37 (2003)),

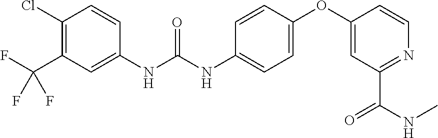

(BAY43-9006; sorafenib),

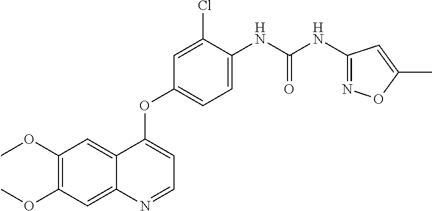

### (KRN951),

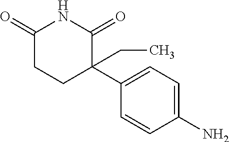

### (Aminoglutethimide);

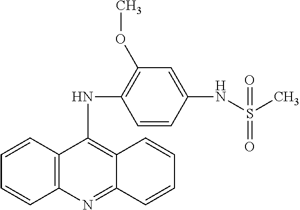

### (Amsacrine);

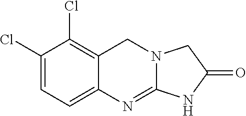

### (Anagrelide);

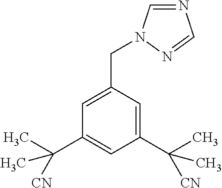

(Anastrozole; sold as Arimidex by AstraZeneca Pharmaceuticals LP; Wilmington, Del.); Asparaginase; Bacillus Calmette-Guerin (BCG) vaccine (Garrido et al., Cytobios. 90(360):47-65 (1997));

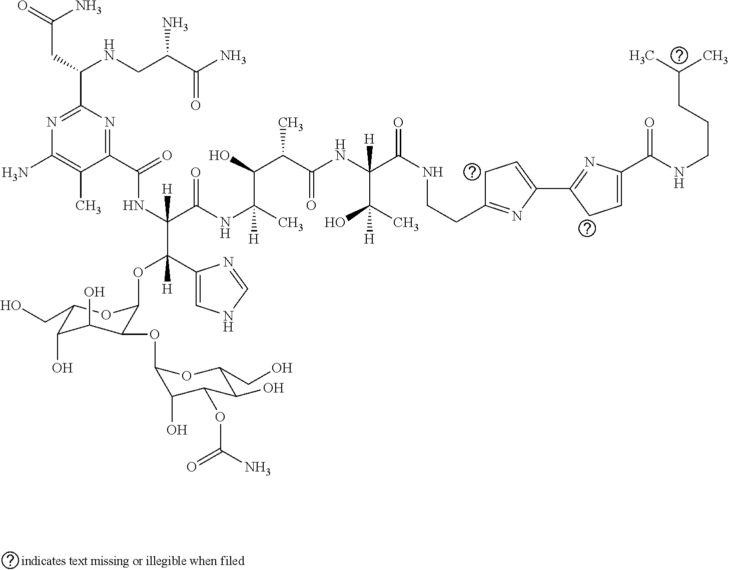

### (Bleomycin);

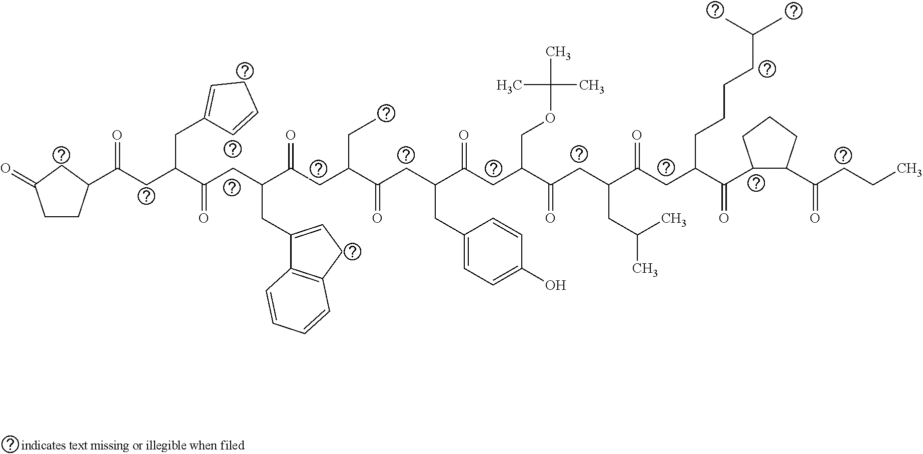

### (Buserelin);

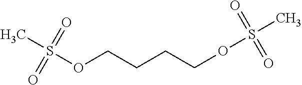

(Busulfan; 1,4-butanediol, dimethanesulfonate; sold as Busulfex® by ESP Pharma, Inc.; Edison, N.J.);

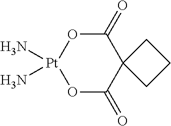

(Carboplatin; sold as Paraplatin® by Bristol-Myers Squibb; Princeton, N.J.);

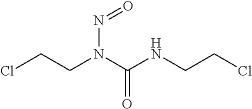

### (Carmustine);

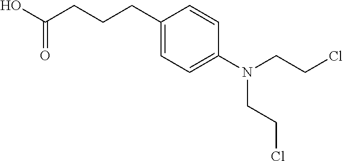

### (Chlorambucil);

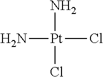

### (Cisplatin):

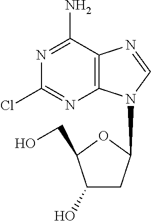

### (Cladribine);

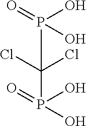

### (Clodronate);

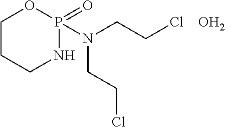

### (Cyclophosphamide);

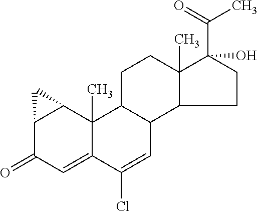

### (Cyproterone);

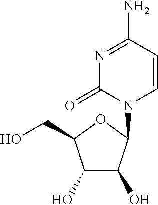

### (Cytarabine);

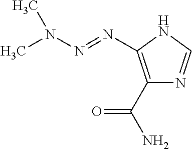

### (Dacarbazine);

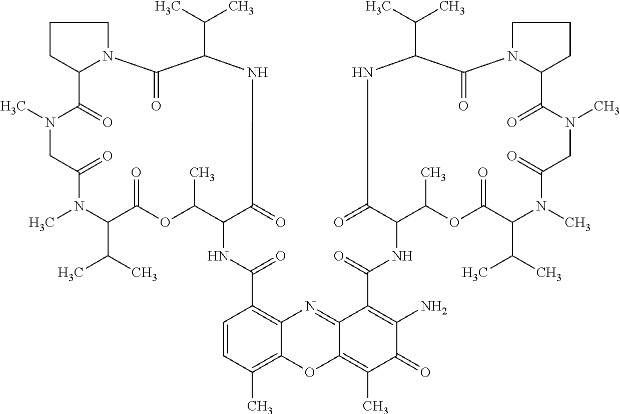

### (Dactinomycin);

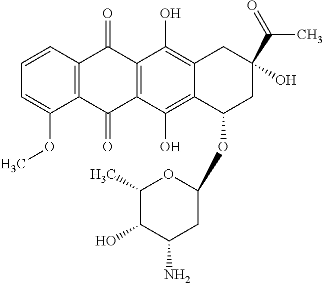

### (Daunorubicin);

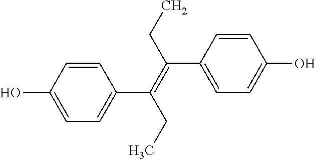

### (Diethylstilbestrol);

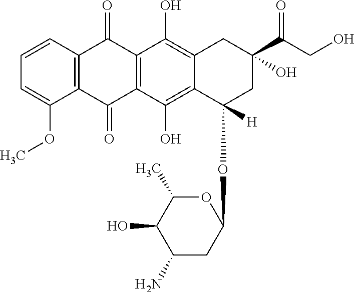

### (Epirubicin);

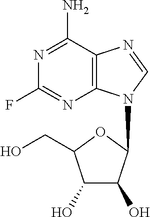

### (Fludarabine);

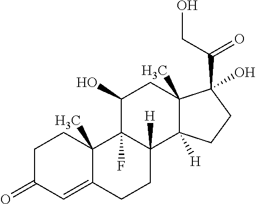

### (Fludrocortisone);

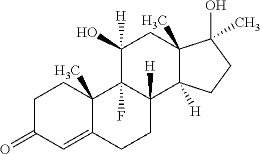

### (Fluoxymesterone);

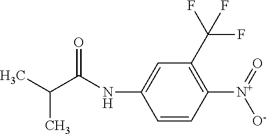

### (Flutamide);

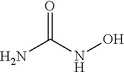

### (Hydroxyurea);

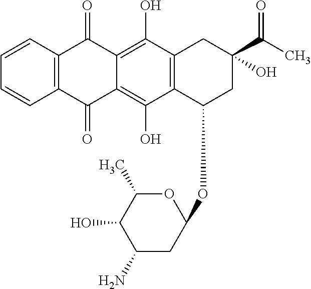

### (Idarubicin);

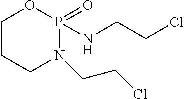

### (Ifosfamide);

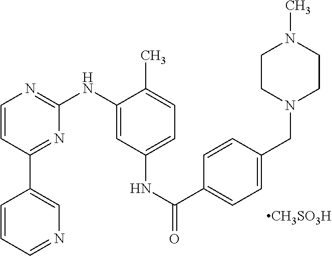

(Imatinib; sold as Gleevec® by Novartis Pharmaceuticals Corporation; East Hanover, N.J.);

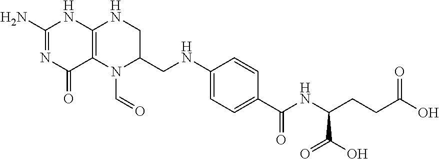

### (Leucovorin);

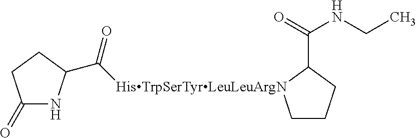

### (Leuprolide);

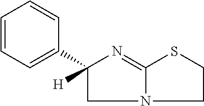

### (Levamisole);

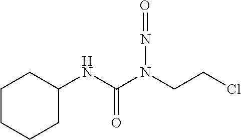

### (Lomustine);

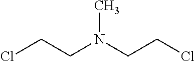

### (Mechlorethamine);

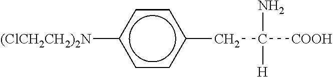

(Melphalan; sold as Alkeran® by Celgene Corporation; Warren, N.J.);

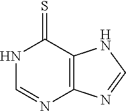

### (Mercaptopurine);

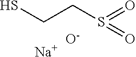

### (Mesna);

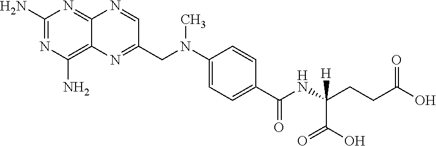

### (Methotrexate);

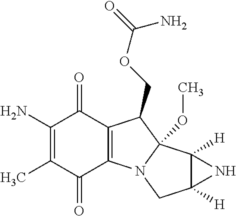

### (Mitomycin);

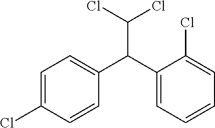

### (Mitotane);

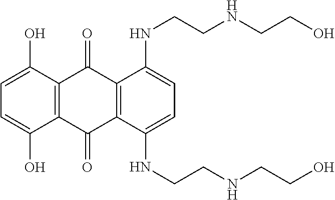

### (Mitoxantrone);

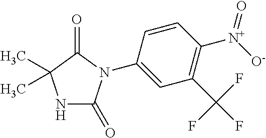

(Nilutamide); octreotide

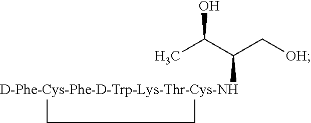

Katz et al., Clin Pharm. 8(4):255-73 (1989); sold as Sandostatin LAR® Depot; Novartis Pharm. Corp; E. Hanover, N.J.); edotreotide (yttrium-90 labeled or unlabeled); oxaliplatin (

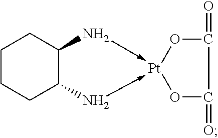

sold as Eloxatin™ by Sanofi-Synthelabo Inc.; New York, N.Y.);

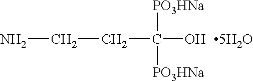

(Pamidronate; sold as Aredia® by Novartis Pharmaceuticals Corporation; East Hanover, N.J.);

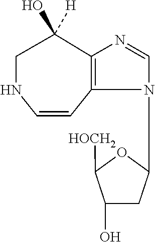

(Pentostatin; sold as Nipent® by Supergen; Dublin, Calif.);

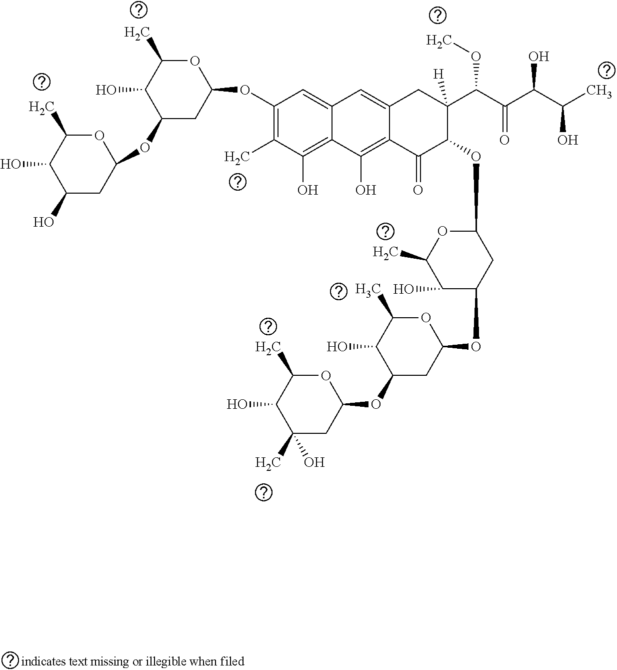

### (Plicamycin);

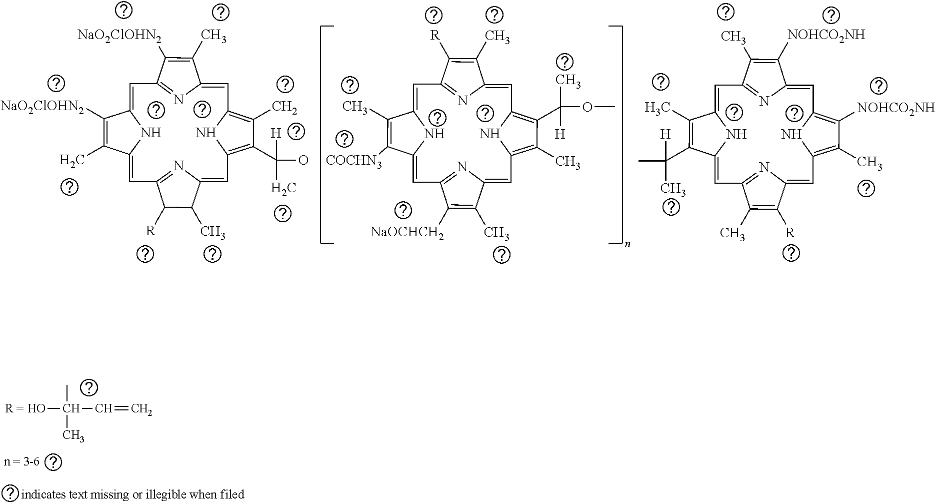

(Porfimer; sold as Photofrin® by Axcan Scandipharm Inc.; Birmingham, Ala.);

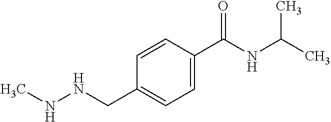

### (Procarbazine);

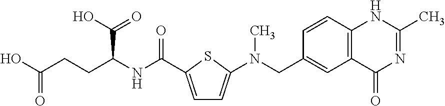

(Raltitrexed); Rituximab (sold as Rituxan® by Genentech, Inc.; South San Francisco, Calif.);

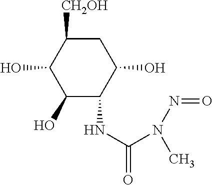

### (Streptozocin);

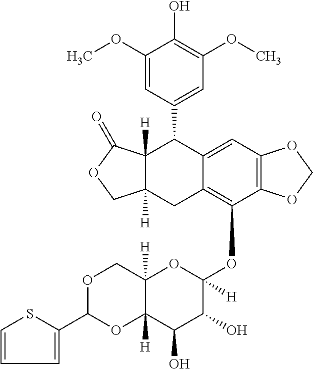

### (Teniposide);

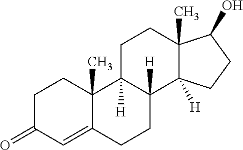

### (Testosterone);

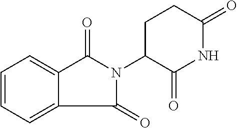

### (Thalidomide);

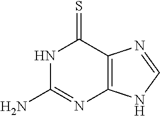

### (Thioguanine);

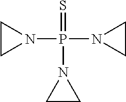

### (Thiotepa);

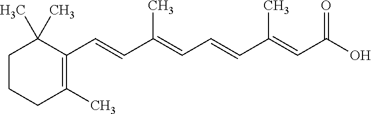

### (Tretinoin);

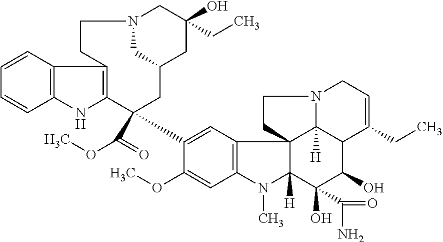

(Vindesine) or 13-cis-retinoic acid

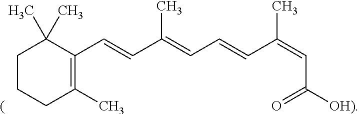

In an embodiment of the invention, an antibody or antigen-binding fragment thereof of the invention is provided in association with one or more of any of: phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), any mTOR inhibitor, rapamycin

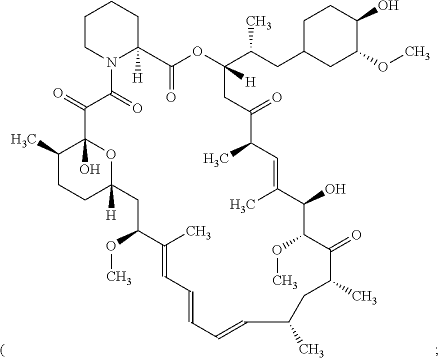

sirolimus), 40-O-(2-hydroxyethyl)-rapamycin, CCI-779

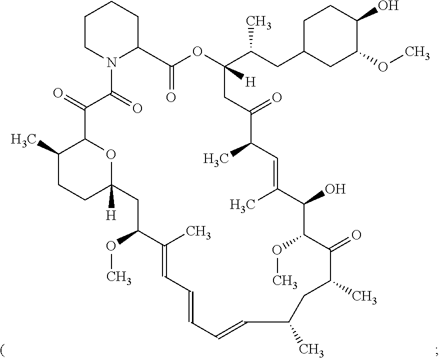

temsirolimus; Sehgal et al., Med. Res. Rev., 14:1-22 (1994); Elit, Curr. Opin. Investig. Drugs 3(8):1249-53 (2002)), AP-23573

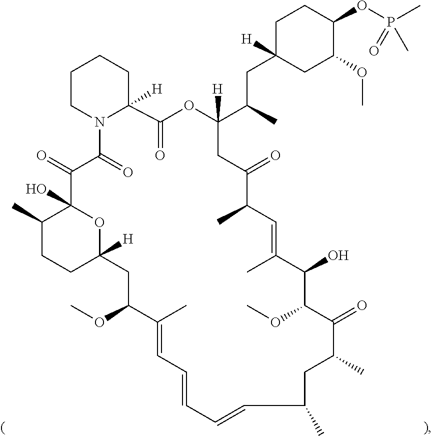

### RAD001

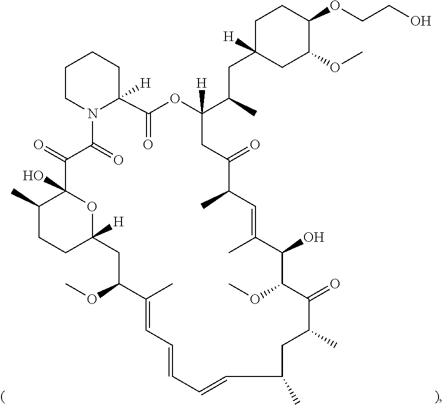

### ABT-578

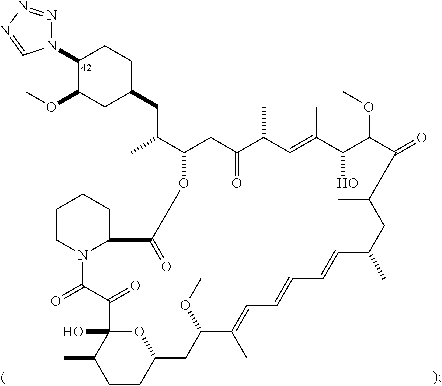

### BC-210

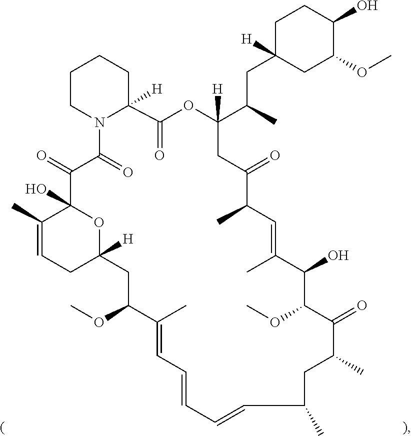

LY294002, LY292223, LY292696, LY293684, LY293646 (Vlahos et al., J. Biol. Chem. 269(7): 5241-5248 (1994)), wortmannin, BAY-43-9006, (Wilhelm et al., Curr. Pharm. Des. 8:2255-2257 (2002)), ZM336372, L-779,450, any Raf inhibitor disclosed in Lowinger et al., Curr. Pharm Des. 8:2269-2278 (2002); flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)).

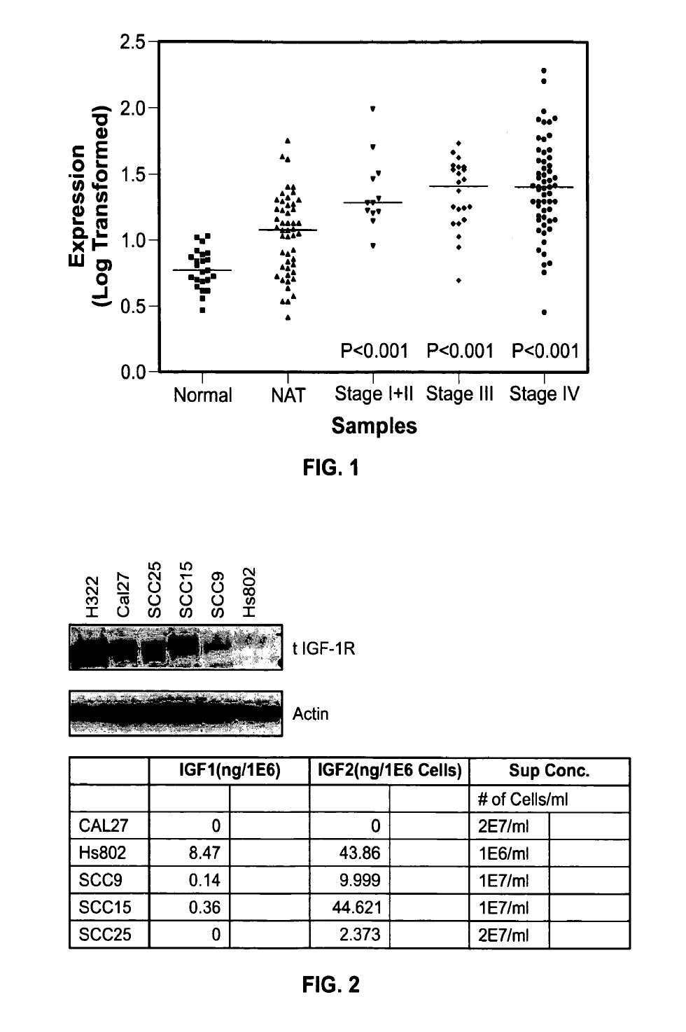

In an embodiment of the invention, an antibody or antigen-binding fragment thereof of the invention is provided in association with one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity;in Fry et al., Science 265 1093-1095 (1994) which discloses a compound having a structure that inhibits EGFR (see FIG. 1 of Fry et al.); in U.S. Pat. No. 5,196,446 which discloses heteroarylethenediyl or heteroarylethenediylaryl compounds that inhibit EGFR; in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylarnino)-8-methyl-8H-pyrido(2,3-d)pyrimidin-7-one.

In an embodiment of the invention, an antibody or antigen-binding fragment thereof of the invention is provided in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4,897,471 and 4,695,623 (especially Examples 7, 8 or 9 thereof), or interferon alpha-n3, a mixture of natural alpha interferons.

Interferon alfa-2a is sold as ROFERON-A® by Hoffmann-La Roche (Nutley, N.J).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “12,000 molecular weight polyethylene glycol conjugated interferon alpha” and “PEG 12000-IFN alpha” as used herein include conjugates such as are prepared according to the methods of International Application No. WO 95/13090 and EP1039922and containing urethane linkages between the interferon alpha-2a or -2b amino groups and polyethylene glycol having an average molecular weight of 12000. The pegylated inteferon alpha, PEG 12000-IFN-alpha-2b is available from Schering-Plough Research Institute, Kenilworth, N.J.

The preferred PEG 12000-interferon alpha-2b can be prepared by attaching a PEG polymer to the histidine residue in the interferon alpha-2b molecule. A single PEG 12000 molecule can be conjugated to free amino groups on an IFN alpha-2b molecule via a urethane linkage. This conjugate is characterized by the molecular weight of PEG 12000 attached. The PEG 12000-IFN alpha-2b conjugate can be formulated as a lyophilized powder for injection.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J).

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. No. 4,766,106, U.S. Pat. No. 4,917,888, European Patent Application No. 0 236 987 or 0 593 868 or International Publication No. WO 95/13090. The preferred PEG12000-IFN alfa 2b can be prepared by attaching in a PEG polymer to a histidine residue in the interferon alfa-2b molecule.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos, 4,492,537; 5,762,923 and 5,766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET® Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes methods of treatment or prevention of a medical condition or disease as set forth herein by administering a composition comprising an anti-IGF1R antibody or antigen-binding fragment thereof of the invention in association with one or more other anti-cancer chemotherapeutic agents (e.g., as described herein) and/or in association with one or more antiemetics including, but not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention includes methods for treating or preventing cancer in a subject by administering an antibody or antigen-binding fragment thereof of the invention optionally in association with one or more other chemotherapeutic agents (e.g., as described herein) and/or optionally in association with one or more antiemetics.

Other side effects of cancer treatment include red and white blood cell deficiency. Accordingly, the present invention includes compositions comprising an anti-IGF1R antibody or antigen-binding fragment thereof optionally in association with an agent which treats or prevents such a deficiency, such as, e.g., pegfilgrastim, erythropoietin, epoetin alfa or darbepoetin alfa.

Another side-effect of anti-cancer therapy is mucositis. Accordingly, the present invention provides methods and compositions for treating or preventing a medical condition or disease as set forth herein by administering an anti-IGF1R antibody or antigen-binding fragment thereof in association with an anti-mucositis treatment. For example, mucositis treatments include swishing and gargling the anesthetic gel viscous Xylocaine® (lidocaine) 2%; or mouth washes including allopurinol, benzydamine hydrochloride, corticosteroids and/or chamomile.

The present invention further comprises a method for treating or preventing any stage or type of any medical condition set forth herein by administering an antibody or antigen-binding fragment thereof of the invention in association with a therapeutic procedure such as surgical tumorectomy or anti-cancer radiation treatment; optionally in association with a further chemotherapeutic agent and/or antiemetic, for example, as set forth above.

The term “in association with” indicates that the components of a composition of the invention (e.g., anti-IGF1R antibody or antigen-binding fragment thereof along with docetaxel) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., wherein an anti-IGF1R antibody is administered parenterally and gefitinib is administered orally).

### Therapeutic Methods and Administration

The present invention includes methods for using an IGF1R inhibitor, which is optionally in association with a further chemotherapeutic agent, or a pharmaceutically formulation thereof, for treating or preventing any of head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia (e.g., T-cell lineage, B-cell precursor lineage), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors, liver cancer, gastric cancer, gigantism, pituitary adenoma, psoriasis and rhabdoid tumor of the kidney.

Pharmaceutical compositions comprising an IGF1R inhibitor in association with a further chemotherapeutic agent (e.g., as set forth above) and a pharmaceutically acceptable carrier are also within the scope of the present invention (e.g., in a single composition or separately in a kit). The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York. In an embodiment of the invention, the antibody is administered to a subject as part of a pharmaceutical composition comprising an antibody, such as LCF/HCA (e.g., 1, 5, 10, 15 20 or 25 mg/ml), sodium acetate trihydrate (e.g., 1.8, 2.3 or 3.1 mg/ml), glacial acetic acid (e.g., 0.5, 0.18 or 2.2 mg/ml); sucrose (e.g., 50 or 70 mg/ml); and water at a pH of about 5.5.

The term “head and neck cancer” and the like includes any cancer occurring in the head and neck area of the body, including the oral cavity, pharynx, paranasal sinuses; nasal cavity, larynx, and salivary glands (e.g., parotid, submanidular, sublingual glands). A common type of head and neck cancer is squamous cell carcinoma occurring in the head and neck area. Other types of head and neck cancers include salivary gland tumors, melanomas, lymphomas and sarcomas.

The term “squamous cell carcinoma” (SCC) and the like includes any cancer involving the squamous cells of the skin. The term includes any subtype of squamous cell carcinoma including, for example, keratoacanthoma, carcinoma cuniculatum, invasive SCC, in situ SCC, or metastatic SCC.

The term “multiple myeloma” and “solitary plasmacytoma” and the like includes cancer of the plasma cell. Solitary plasmacytoma is myeloma in a single place in the body. The term “multiple myeloma” includes embodiments wherein, e.g., myeloma cells occur in multiple sites in the body (e.g., bone marrow sites). The term includes any subtype of multiple myeloma including, for example, light chain myeloma, non-secretory myeloma,

The term “renal cell carcinoma” and the like is also called cancer of the kidney or renal adenocarcinoma and includes embodiments wherein cancer cells are found in any tissues of the kidney. The term includes, for example, any subtype of renal cell carcinoma including, for example, clear cell carcinomas (mixed with granular cells or not), chromophilic cancers, rhabdoid tumors of the kidney, chromophobic cancers, oncocytic cancers, collecting duct cancers, transitional cell carcinomas and sarcomatoid tumors.

The term “subject” or “patient” includes any organism, preferably a mammal (e.g., primate, dog, horse, rat, mouse, cat, rabbit) and most preferably a human. In an embodiment of the invention, a “subject” or “patient” is an adult human (e.g., 18 years or older) or a human child (e.g., under 18 years of age, for example, less than 1, 1, 2, 3, 4, 5, 6, 7,8, 9 or 10 years of age).

A pharmaceutical composition containing an IGF1R inhibitor, which is optionally in association with a further chemotherapeutic agent, can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular, topical, intra-peritoneal, inhalation, intra-cranial) and non-parenteral (e.g., oral, transdermal, intranasal, intraocular, sublingual, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients include, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment of the invention, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN-80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

In an embodiment of the invention, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

Implantation of a slow-release or sustained-release system, such that a constant level of dosage is maintained is also contemplated herein. Briefly, an active agent (e.g., IGF1R inhibitor, which is optionally in association with a further chemotherapeutic agent) is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the IGF1R inhibitor, which is optionally in association with a further chemotherapeutic agent, and the needs of the subject.

The concentration of the IGF1R inhibitor, which is optionally in association with a further chemotherapeutic agent, can be adjusted so that an injection provides an effective amount to produce the desired pharmacological effect. As discussed below, the exact dose depends on the age, weight and condition of the patient or animal as is known in the art.

In an embodiment of the invention, unit-dose parenteral preparations are packaged in an ampoule, a vial or a syringe with a needle. All preparations for parenteral administration must be sterile, as is known and practiced in the art.

In an embodiment of the invention, an IGF1R inhibitor, which is optionally in association with a further chemotherapeutic agent, is formulated into a lyophilized powder, which can be reconstituted for administration as solutions, emulsions and other mixtures. The powder may also be reconstituted and formulated as a solid or gel.

In an embodiment of the invention, the sterile, lyophilized powder is prepared by dissolving an IGF1R inhibitor (e.g., an anti-IGF1R antibody), which is optionally in association with a further chemotherapeutic agent, or a pharmaceutically acceptable derivative thereof, in a suitable solvent. The solvent may contain an excipient which improves the stability or other pharmacological components of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the IGF1R inhibitor optionally in association with the further chemotherapeutic agent. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In an embodiment of the invention, for reconstitution, the lyophilized powder is added to sterile water or other suitable carrier. The precise amount depends upon the selected therapy being given. Such amount can be empirically determined.

Administration by inhalation can be provided by using, e.g., an aerosol containing sorbitan trioleate or oleic acid, for example, together with trichlorofluoromethane, dichlorofluoromethane, dichlorotetrafluoroethane or any other biologically compatible propellant gas; it is also possible to use a system containing an IGF1R inhibitor, which is optionally in association with a further chemotherapeutic agent, by itself or associated with an excipient, in powder form.

In an embodiment of the invention, IGF1R inhibitor, which is optionally in association with a further chemotherapeutic agent, is formulated into a solid dosage form for oral administration, in one embodiment, into a capsule or tablet. Tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

Any of the agents set forth herein can be formulated into a sustained release formulation including liposomal formulations such as unilamellar vesicular (ULV) and multilamellar vesicular (MLV) liposomes and DepoFoam™ particles (Kim et al., Biochim. Biophys. Acta (1983) 728(3):339-348; Kim, Methods Neurosci. (1994) 21: 118-131; Kim et al., Anesthesiology (1996) 85(2): 331-338; Katre et al., J. Pharm. Sci. (1998) 87(11): 1341-1346). A feature of the DepoFoam system is that, inside each DepoFoam particle, discontinuous internal aqueous chambers, bounded by a continuous, non-concentric network of lipid membranes render a higher aqueous volume-to-lipid ratio and much larger particle diameters compared with MLV.

### Dosage and Administration

Methods of the present invention include administration of an IGF1R inhibitor, which is optionally in association with a further chemotherapeutic agent, or a pharmaceutical composition thereof. Typically, the administration and dosage of such agents is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

The term “therapeutically effective amount” or “therapeutically effective dosage” means that amount or dosage of a composition of the invention (e.g., IGF1R inhibitor, such as an anti-IGF1R antibody) that will elicit a biological or medical response of a tissue, system, subject or host that is being sought by the administrator (such as a researcher, doctor or veterinarian) which includes any measurable alleviation of the signs, symptoms and/or clinical indicia of cancer (including e.g., inhibition of any IGF1R activity such as IGF-I or IGF-II binding or kinase activity), such as head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia (e.g., T-cell lineage, B-cell precursor lineage), acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors and liver cancer (e.g., tumor growth or the proliferation of cancerous cells) and/or the prevention, slowing or halting of progression or metastasis of the cancer to any degree. For example, in one embodiment, a “therapeutically effective dosage” of any anti-IGF1R antibody; for example, an antibody or antigen-binding fragment thereof comprising (a) a light chain variable region comprising amino acids 20-128 of SEQ ID NO: 2 and a heavy chain variable region comprising amino acids 20-137 of SEQ ID NO: 10 or 12; (b) a light chain variable region comprising amino acids 20-128 of SEQ ID NO: 4 and a heavy chain variable region comprising amino acids 20-137 of SEQ ID NO: 10 or 12; (c) a light chain variable region comprising amino acids 20-128 of SEQ ID NO: 6 and a heavy chain variable region comprising amino acids 20-137 of SEQ ID NO: 10 or 12; or (d) a light chain variable region comprising amino acids 20-128 of SEQ ID NO: 8 and a heavy chain variable region comprising amino acids 20-137 of SEQ ID NO: 10 or 12; or any other anti-IGF1R antibody mentioned herein is between about 0.3 and about 20 mg/kg of body weight (e.g., about 0.5 mg/kg body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), twice per week, once per week, once every 2 weeks, once every 3 weeks or once a month.

Dosage regimens may be adjusted to provide the optimum desired response (e.g., a therapeutic response). For example, a single dose may be administered or several divided doses may be administered over time or the dose may be proportionally reduced or increased as indicated by exigencies of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. It is especially advantageous to formulate parenteral compositions in dosage unit form for ease of administration and uniformity of dosage.

A practitioner (e.g., physician or veterinarian) having ordinary skill in the art can readily determine and prescribe the effective amount of the pharmaceutical composition required. For example, the physician or veterinarian could start doses of the antibody or antigen-binding fragment of the invention employed in the pharmaceutical composition at levels lower than that required in order to achieve the desired therapeutic effect and gradually increase the dosage until the desired effect is achieved. The effectiveness of a given dose or treatment regimen of an antibody or combination of the invention can be determined , for example, by determining whether a tumor being treated in the subject shrinks or ceases to grow. The size of tumor can be easily determined, for example, by X-ray, magnetic resonance imaging (MRI), visually in a surgical procedure or manually by palpation. Tumor size and proliferation can also be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124I]iodo-2′-deoxyuridine), [⁷⁶Br]BrdUrd (Bromodeoxyuridine), [¹⁸F]FLT (3′-deoxy-3′fluorothymidine) or [¹¹C]FMAU (2′-fluoro-5-methyl-1-β-D-arabinofuranosyluracil).

For example, head and neck cancer progress can be monitored, by the physician or veterinarian by a variety of methods, and the dosing regimen can be altered accordingly. Methods by which to monitor head and neck cancer include, for example, physical examination (e.g., visual or tactile examination), endoscopy of hard to observe areas (e.g., nasopharyngoscopy, pharyngoscopy, or laryngoscopy), computed tomographic scan (CT), magnetic resonance image scan (MRI), ultrasound exam, positron emission tomography (PET) scans, panorex (X-ray of the jaws), barium swallow, dental X-rays, chest X-rays, and radionuclide bone scan.

For example, squamous cell carcinoma progress can be monitored, by the physician or veterinarian by a variety of methods, and the dosing regimen can be altered accordingly. Methods by which to monitor squamous cell carcinoma include, for example, by patient interview or by physical examination (e.g., visual inspection and documentation of any lesion's size, shape or other visual qualities).

For example, multiple myeloma progress can be monitored, by the physician or veterinarian by a variety of methods, and the dosing regimen can be altered accordingly. Methods by which to monitor multiple myeloma include, for example, with a complete blood court (CBC), for example, to detect low hematocrit (anemia), low red blood cell count, low platelets, and/or low white blood cell count, bone marrow biopsy, serum protein electrophoresis, bone X-rays to identify fractures and hollowed out bone lesions, or a chemistry profile to detect increased serum calcium, total protein, or abnormal kidney function.

For example, renal cell carcinoma progress can be monitored, by the physician or veterinarian by a variety of methods, and the dosing regimen can be altered accordingly. Methods by which to monitor renal cell carcinoma include, for example, palpation of the abdomen to detect a mass, complete blood count (CBC), urine test to detect red blood cells, assay of serum calcium levels to detect elevation, assay of Serum glutamate pyruvate transaminase (SGPT) and alkaline phosphatase to detect elevation, urine cytology assay, liver function tests, an ultrasound examination of the abdomen and kidney, kidney X-ray, intravenous pyelogram (IVP) or renal arteriography.

Compositions and methods of the invention include an IGF1R inhibitor optionally “in association” with one or more chemotherapeutic agents. The term “in association” indicates that the components of the combinations of the invention can be formulated into a single composition for simultaneous delivery or formulated separately into two or more compositions (e.g., a kit). Furthermore, each component of a combination of the invention can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., orally, intravenously, subcutaneously).

### EXAMPLES

The present invention is intended to exemplify the present invention and not to be a limitation thereof. Any method or composition disclosed below falls within the scope of the present invention.

### Example 1

### IGF1R and IGF-II are Expressed in Head and Neck Cancer Cells

In this example, head and neck cancer cells were assayed to determine the level of expression of IGF1R or IGF-2. Primary tumor tissue samples (from stage 2, 3 or 4 cancers), normal adjacent tissue samples and normal tissue samples were analyzed for the level of IGF1R RNA expression. Furthermore, the level of IGF1R, IGF-1 and IGF-2, in various cell lines, were analyzed.

RNA was made from tumor samples and cDNAs were synthesized therefrom. Expression of IGF1R was analyzed using a 20 ng cDNA sample in a Fluorogenic 5′-nuclease PCR assay with specific probes and primers using the ABI Prism 7700 Sequence detection System 9 (Applied Biosystems; Foster City, Calif.). CT numbers were normalized against ubiqitin or actin mRNA expression in all samples.

The primers and probes used were as follows:

(SEQ ID NO: 106)
IGF1R/forward primer: GAAAGTGACGTCCTGCATTTCA
(SEQ ID NO: 107)
IGF1R/reverse Primer: CCGGTGCCAGGTTATGATG
(SEQ ID NO: 108)
Probe Sequence: CACCACCACGTCGAAGAATCGCA

H322 is a non-small cell lung cancer cell line; CAL27, SCC25, SCC15, SCC9 and HS802 are squamous cell carcinoma cell lines.

The data generated in these methods is set forth in FIGS. 1 and 2. FIG. 1 demonstrates that IGF1R was expressed, in primary head and neck tumor samples, at a higher level than that of normal tissue. Moreover, normal adjacent tissue (NAT) exhibited a greater level of IGF1R expression than that of normal tissue. The level of IGF1R expression increased slightly as the stage of head and neck tumor increased (from I to IV). Each point represents the normalized level of IGF1R in a single tissue sample.

FIG. 2 also demonstrates that IGF1R, IGF-I and IGF-II were expressed in various lung and squamous cell carcinoma cell lines. The top panel is a western blot analysis of several cell lines demonstrating expression levels of total IGF1R (tIGF-1R) on the cell. Actin expression is also shown as an internal control. The bottom panel indicates the level of IGF1 and IGF2 secreted secreted from the cells assayed (ng secreted protein per 10⁶ cells).

### Example 2

### Anti-IGF1R (LCF(κ)/HCA(γ1)) Inhibits in vitro Proliferation of Squamous Cell Carcinoma Cell Lines

This example demonstrates that the anti-IGF1R antibody LCF/HCA inhibits proliferation of various squamous cell carcinoma (SCC) cell lines.

Cell proliferation was assayed using a Cell-Titer Glo assay (Promega Corp.; Madison, Wis.). A cell-titer glo assay assay generates a “glow” luminescent signal in the presence of ATP from viable cells, which can be detected with a plate reader luminometer or CCD imaging device.

In this assay, squamous cell carcinoma cells, SCC9, SCC25 and SCC15, were trypsinized, counted and resuspended at 25,000 cells/ml in 10% HI-FBS RPMI containing NEAA, L-Glu, MEM Vitamins and PS. 100 ml of cell suspension (2500 cells) were added to each well BD Falcon 96 well black, clear bottom TC treated plate. Cells were allowed to attach and spread overnight at 37° C. Then, 10% RPMI medium was replaced with 100m1 2% RPMI, containing the LCF/HCA antibody at the appropriate concentration, the following day. The anti-IGF1R antibody concentrations used in each experiment were 100 nM, 20 nM, 5 nM, 0.8 nM, 0.16 nM, 0.032 nM, 0.0064 nM, 0.00128 nM, and 0.000256 nM. All treatments were prepared in 2% RPMI at 20× concentration and serial diluted. Every test point was prepared in triplicate on separate assay plates. As mentioned above, cell proliferation was measured using the CellTiter-Glo Luminescent Cell Viability Assay (Promega) at 96 hours post treatment. Luminescence was detected on a Wallac 420 Plate Reader with stacker.

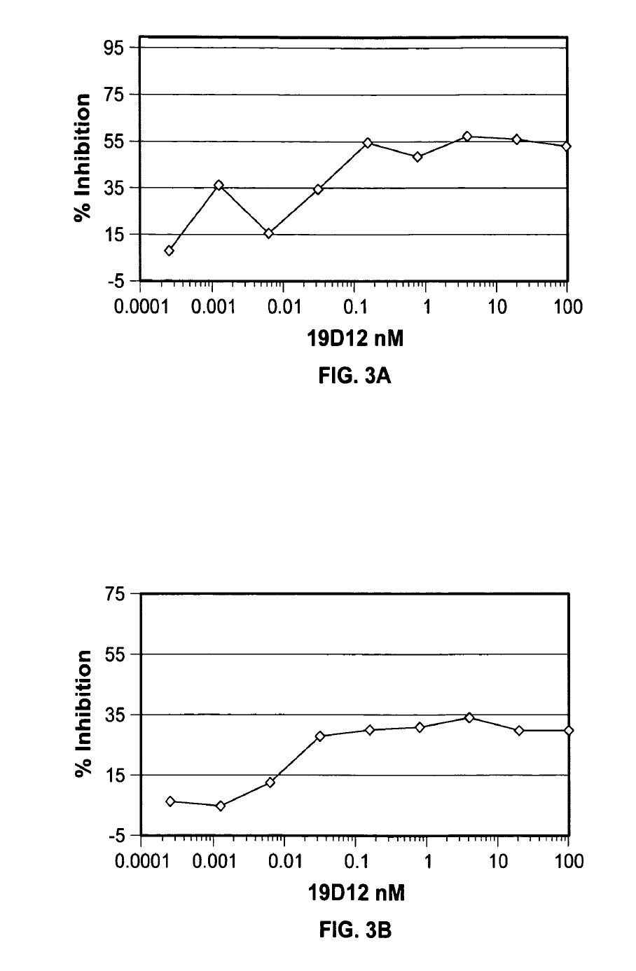

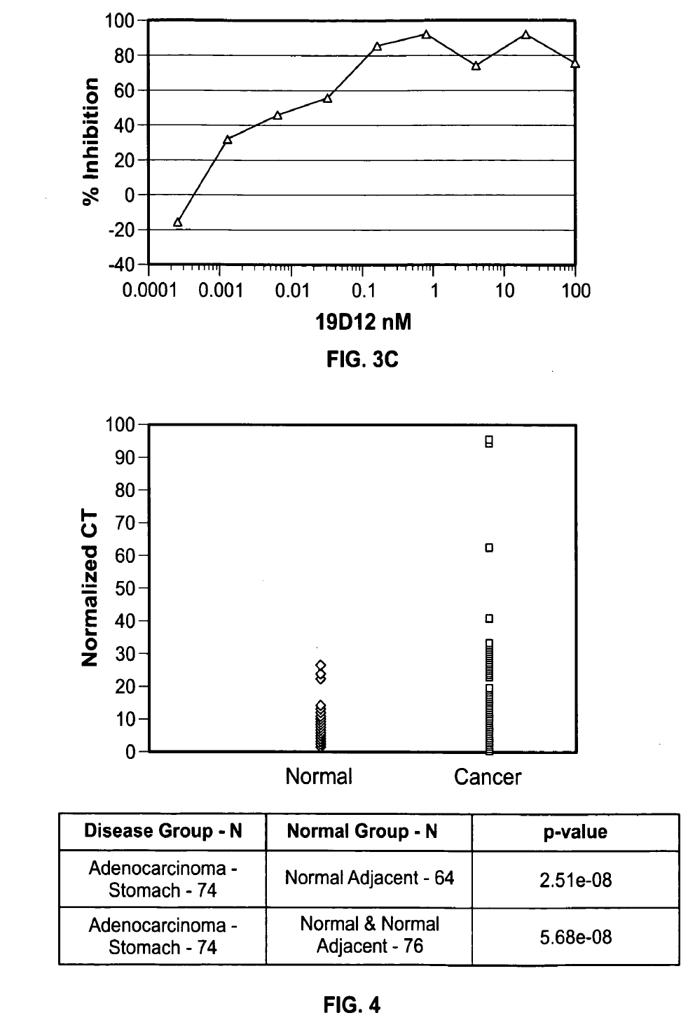

The results of this work is set forth in FIGS. 3a-3c which demonstrates that proliferation of squamous cell carcinoma cell lines was inhibited by exposure to various concentrations of LCF/HCA (indicated in the figures as 19D12). The data in figures (a), (b), and (c) correspond to the cell lines discussed above as follows: (a): cell line SCC 15; (b): cell line SCC 25; (c): cell line SCC 9.

### Example 3

### IGF2 and IGF1R are Highly Expressed in Gastric and Ovarian Cancer Cell Lines

In this example, IGF2 mRNA was demonstrated to be expressed at a high level in primary gastric tumor samples and in stomach adenocarcinoma cell lines as compared to normal samples and cell lines. High expression of IGF2 in a tumor sample is a marker for autocrine stimulation through the IGF1R growth pathway and indicates that the tumor's growth will be inhibited by anti-IGF1R (LCF(κ)/HCA(γ1)) antibody treatment.

Cell lines in which expression levels were to be determined were grown, prepared and assayed essentially as set forth above. Protein levels were analyzed by western blot and mRNA levels were analyzed by Taqman analysis. Quantitative flow cytometry was conducted using cells labeled with anti-IGF1R, IR (insulin receptor), EGFR (epidermal growth factor receptor) or HER2 antibodies using the Quantitative Simply Cellular system of Bangs Laboratories, Inc. (Fishers, Ind.).

A2780 is a human ovarian cancer cell line and NCI-N87, SNU-16, SNU-1 and Hs746T are human gastric cancer cell lines.

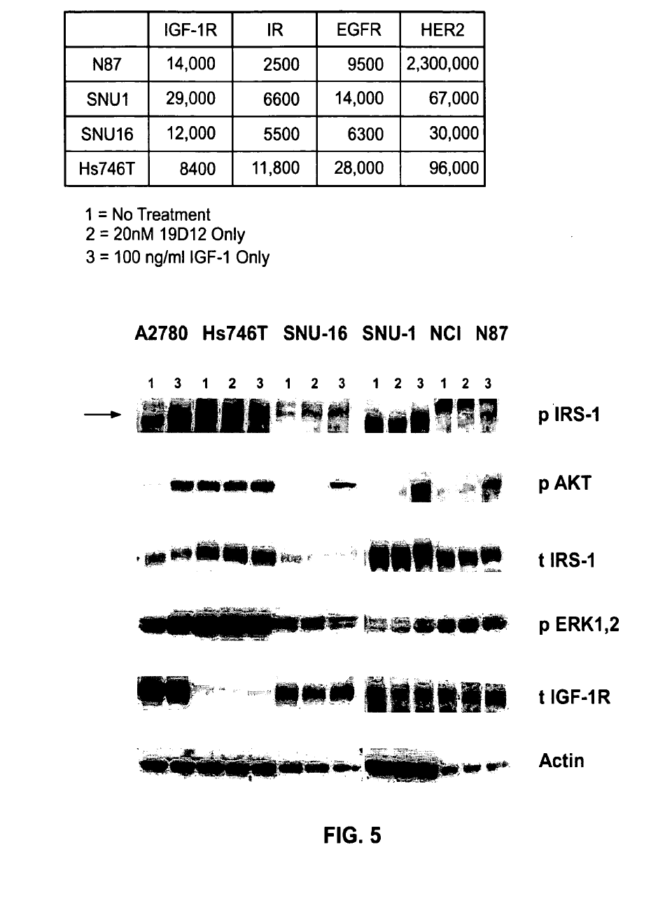

The results of this work are set forth in FIGS. 4 and 5. FIG. 4 demonstrates that IGF2 was expressed at high levels in primary gastric tumor cells as compared to that of normal cells and normal adjacent cells. Each point is the normalized level of IGF2 mRNA expressed in a single tumor tissue sample (normalized against the expression levels of ubiquitin).

The FIG. 5 top panel is quantitative flow cytometry data, relating to several cell lines, indicating the number of receptors/cell for IGF1R, IR, EGFR and HER2. The FIG. 5 bottom panel is western blot data for several cell lines demonstrating the expression level of pIRS-1 (phosphorylated insulin receptor substrate-1 (IRS-1)), pAKT (phosphorylated AKT (also known as protein kinase-B) (non-isoform specific)), tIRS-1 (total IRS-1), pERK1, 2 (phosphorylated ERK1 (extracellular signal-regulated kinase 1) and ERK2 (extracellular signal-regulated kinase 2)), tIGR1 R (total IGFR1) and actin. The band under lane 3 of NCI N87 in the t IRS-1 row migrates slightly slower than expected suggesting that it is HER2 which cross reacts with the anti-IRS-1 antibodies used in this experiment.

### Example 4

### Anti-IGF1R (LCF(κ)/HCA(γ1)) Antibody Inhibits in vitro Proliferation of Gastric Carcinoma and Myeloma Cell Lines

This example demonstrates that proliferation of the gastric cancer cell line SNU-16 cells and the myeloma cell line RPMI8226 was inhibitied by the anti-IGF1R antibody LCF/HCA. Cells were grown, prepared and assayed using the Cell-Titer Glo assay essentially as set forth above. The anti-IGF1R antibody concentrations used in each experiment were 100 nm, 20 nm, 5 nm, 0.8 nm, 0.16 nm, 0.032 nm, 0.0064 nm, 0.00128 nm, and 0.000256 nM

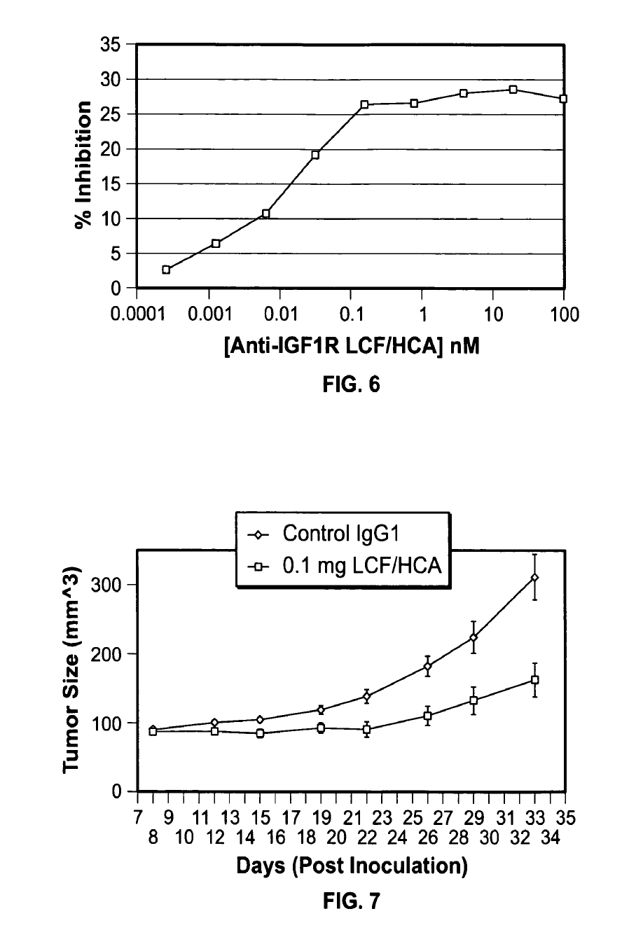

The results of this work are set forth in FIG. 6. FIG. 6 demonstrates in vitro growth inhibition of the SNU-16 cell line at several concentrations of the antibody. A 40%-45% level of growth inhibition of the myeloma cell line was also observed in vitro.

### Example 5

### Anti-IGF1R (LCF(κ)/HCA(γ1)) Antibody Inhibits in vivo Renal Cell Carcinoma and in vitro Melanoma Tumor Cell Growth

This example demonstrates the efficacy of anti-IGF1R therapy for the treatment or prevention of renal cell cancer or melanoma.

Athymic nude mice were inoculated with A498 human kidney carcinoma tumor cells in the right flank, subcutaneously, along with Matrigel (1:1::cells:gel). In these experiments, 5×10⁶ cells/mouse in a 1:1 mix with regular matrigel. The LCF/HCA anti-IGF1R antibody was inoculated intraperitoneally (ip) (0.1 mg/injection). Mice were dosed with antibody two times per week, by intraperitoneal injection, seven times starting on day 12. Tumor size was measured with calipers and the data was entered into the LABCAT data experimental collection and analysis program program (Innovative Programming Associates, Inc.; Princeton, N.J.). Mice were grouped with average size of 100 mm³. Tumor size and body weight were measured twice weekly.

Human melanoma cells (A375-SM) were also assayed in vitro for responsiveness to anti-IGF1R antibody at 100 nM, 20 nM, 5 nM, 0.8 nM, 0.16 nM, 0.032 nM, 0.0064 nM, 0.00128 nM, and 0.000256 nM concentrations. Assays were conducted as set forth above by Cell-Titer Glo assay.

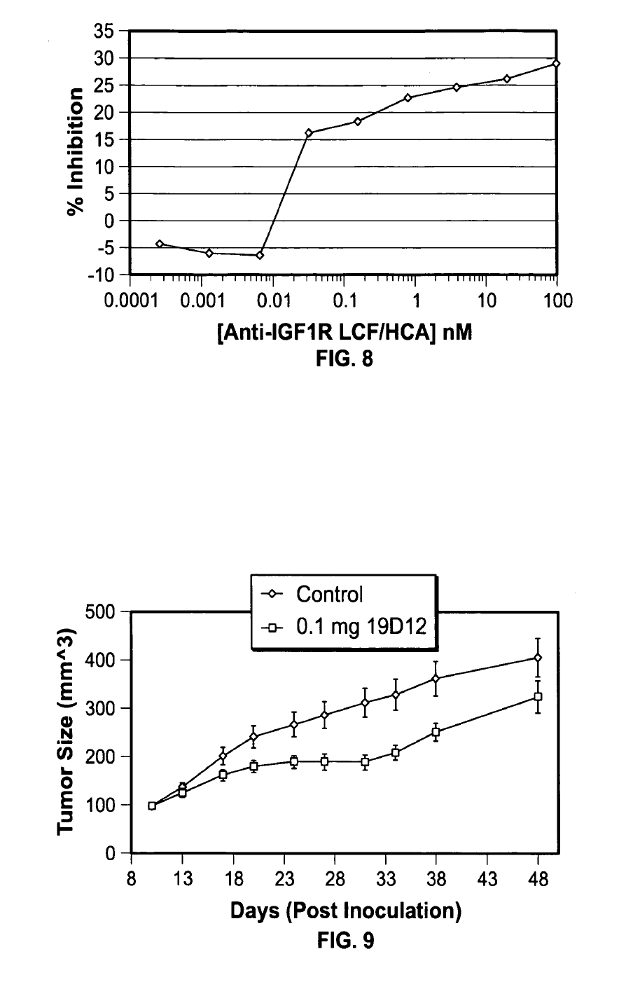

The results of this work are set forth in FIGS. 7 and 8. FIG. 7 demonstrates in vivo growth inhibition of the renal cell carcinoma cell line over time when the LCF/HCA antibody was administered. FIG. 8 demonstrates the in vitro growth inhibition of the melanoma cell line at various concentions of LCF/HCA antibody.

### Example 6

### Anti-IGF1R (LCF(κ)/HCA(γ1)) Antibody Inhibits in vivo Squamous Cell Carcinoma Cell Growth

This example demonstrates the efficacy of anti-IGF1R therapy for the treatment or prevention of head & neck cancers such as squamous cell carcinoma.

Immune-deficient SCID mice (strain SCID) were inoculated with SCC15 human squamous cell carcinoma tumor cells in the right flank, subcutaneously, along with Matrigel (1:1 cells:gel). In these experiments, 5×10⁶ cells/mouse in a 1:1 mix with regular matrigel were inoculated. The LCF/HCA anti-IGF1R antibody was inoculated intraperitoneally (ip) (0.1 mg/injection). Mice were dosed with the antibody two times per week, by intraperitoneal injection, seven times starting on day 10 with the last dose on day 31. Tumor size was measured with calipers and the data was entered into the labcat program. Mice were grouped with average size of 100 mm³. Tumor size and body weight were measured twice weekly.

The data generated from these experiments are set forth in FIG. 9. Tumor growth in the mice inoculated with the LCF/HCA antibody (indicated as “19D12” on the graph) was less than that observed in mice only receiving a control injection without the antibody.

### Example 7

### PPTP Panel Testing of LCF(κ)/HCA(γ1) Anti-IGF1R Antibody

LCF/HCA is a fully human antibody directed against the insulin-like growth factor 1 receptor (IGF1R), which is implicated in the growth and metastatic phenotype of a broad range of malignancies. IGF1R signaling is of particular importance in the childhood cancer setting, e.g., in connection with pediatric cancers such as neuroblastoma, Ewing sarcoma, rhabdomyosarcoma, Wilms tumor, and osteosarcoma. The activity of the antibody was evaluated against the in vitro and in vivo panels of the Pediatric Preclinical Testing Program (PPTP). PPTP includes a panel of in vitro cell line and in vivo mouse xenograft panels, established and organized by the National Cancer Institute (NCI), which allows testing of therapies in the most common pediatric cancers (see e.g., Houghton et al., Pediatric Blood & Cancer (2007) 49(7):928-40).

The PPTP includes a molecularly characterized in vitro panel of cell lines (n=27) and in vivo panel of xenografts (n=61) representing most of the common types of childhood solid tumors and childhood ALL (acute lymphoblastic leukemia). LCF/HCA anti-IGF1R antibody was tested against the PPTP in vitro panel at concentrations ranging from 0.01 nM to 100 nM; and was tested against the PPTP in vivo panel at a dose of 0.5 mg per mouse administered twice weekly for four weeks via intraperitoneal injection. Three measures of antitumor activity were used: 1) response criteria modeled after the clinical setting; 2) treated-to-control (T/C) tumor volume at day 21; and 3) a time to event (4-fold increase in tumor volume) measure based on the median EFS of treated and control lines (intermediate activity required EFS (event-free survival) T/C>2, and high activity additionally required a net reduction in median tumor volume at the end of the experiment).

Methods

In vitro testing. In vitro testing was performed using DIMSCAN, a semiautomatic fluorescence-based digital image microscopy system that quantifies viable cell numbers (using fluorescein diacetate [FDA]) in tissue culture multiwell plates (Keshelava et al., Methods Mol. Med. (2005) 110:139-153). Testing was for 96 hours at concentrations from 0.01 nM to 0.1 mM with replicates of 6 per data point. Data were analyzed using Kaleidagraph software (Synergy Software; Reading, Pa.), fitting a non-linear regression, sigmoidal dose-response model to the response; relative fluorescence values vs. the concentration. The PPTP in vitro panel contains cell lines for neuroblastoma (4), Ewing sarcoma (4), rhabdomyosarcoma (4), acute lymphoblastic leukemia (5), NHL (2), and others.

Stage 1 testing involved testing an agent across the entire PPTP panel of childhood cancer xenograft lines at its maximum tolerated dose (MTD) or at a dose selected based on PK/PD (pharmacokinetic/pharmacodynamic) studies using adult preclinical models.

In vivo solid tumor testing: For each xenograft line, 10 mice bearing subcutaneous (SC) tumors initiated treatment when the tumors were between 0.2-0.5 cm³ in volume. Two perpendicular tumor diameters were measured at once weekly intervals with digital vernier calipers. Assuming tumors to be spherical, volumes were calculated from the formula (π/6)×d3, where d represented the mean diameter.

In vivo acute lymphoblastic leukemia testing: For each xenograft line, 8 mice were inoculated with 3-5×10⁶ mononuclear cells purified from the spleens of secondary recipient mice. Engraftment was monitored weekly by flow cytometry, and treatment was initiated when the proportion of human CD45+ cells in the peripheral blood reached 1%. The proportion of human CD45+ cells in the peripheral blood was monitored weekly throughout the course of treatment.

Drug: LCF/HCA was dissolved in 20mM sodium acetate pH 5 buffer containing 150 mM sodium chloride, and administered intraperitoneally twice weekly for 4 consecutive weeks at a dose of 0.5 mg per animal. The antibody was provided to each testing site in coded vials for blinded testing according the PPTP's standard operating procedures.

### Solid Tumor Response Criteria:

| Response | Definition | Score |
|---|---|---|
| PD1 (Progressive Disease 1) | >25% ↑ in tumor volume, TGD | 0 |
| | value ≦ 1.5 | |
| PD2 (Progressive Disease 2) | >25% ↑ in tumor volume, TGD | 2 |
| | value > 1.5 | |
| SD (Stable Disease) | <25% ↑ in tumor volume, <50% | 4 |
| | regression | |
| PR (Partial Response) | ≧50% regression, but no CR | 6 |
| CR (Complete Response) | <0.1 cm3 tumor volume | 8 |
| MCR (Maintained CR) | <0.1 cm3 tumor volume at the | 10 |
| | end of study | |

### Leukemia Response Criteria:

| Response | Definition | Score |
|---|---|---|
| PD1 (Progressive | No PR & TGD value of ≦ | 0 |
| Disease 1 ) | 1.5 & events at EOS | |
| PD2 (Progressive | No PR & TGD value > | 2 |
| Disease 2) | 1.5 & events at EOS | |
| SD (Stable Disease) | No PR and no events at | 4 |
| | EOS | |
| PR (Partial Response) | CD45% < 1% for only 1 | 6 |
| | week | |
| CR (Complete | CD45% < 1% for 2 | 8 |
| Response) | consecutive weeks | |
| MCR (Maintained CR) | CD45% < 1% for last 3 | 10 |
| | weeks of study | |

Median Group Response: Each individual mouse in each treatment group was assigned a response score (see Tables above) and a median score for the treatment group was calculated and then each treatment group was assigned an overall response according to the table below.

| If Median Score | Overall Group |
|---|---|
| (MS) from (1): | Response |
| 0 ≦ MS ≦ 1 | PD1 |
| 1 < MS ≦ 3 | PD2 |
| 3 < MS ≦ 5 | SD |
| 5 < MS ≦ 7 | PR |
| 7 < MS ≦ 9 | CR |
| 9 < MS | MCR |

Statistical Methods: Event-free survival (EFS) distributions of each treatment group were compared to the EFS distribution of the respective control group using the exact log rank test. P-values were 2-sided & were not adjusted for multiple comparisons given the exploratory nature of this study. P-values<0.05 were considered to be significant.

### Results

TABLE 1
Summary of LCF/HCA in vivo activity

| | | | | | Tumor | | Overall |
|---|---|---|---|---|---|---|---|
| Xenograft | | | EFS | Median Final | Volume | | Group |
| Line | Histology | P-value | T/C | RTV | T/C | P-value | Response |
| BT-29 | Rhabdoid | 0.066 | 1.4 | >4 | 0.77 | 0.247 | |
| KT-16 | Rhabdoid | 0.24 | 1.2 | >4 | 0.5 | 0.130 | |
| KT-14 | Rhabdoid | | >3.3# | 1.6# | 0.6 | | PD2 |
| KT-10 | Wilms | | 1.3 | >4 | 0.43 | | |
| KT-11 | Wilms | 0.547 | 1 | >4 | 0.99 | 1.000 | |
| KT-13 | Wilms | 0.374 | 1.1 | >4 | 0.84 | 0.236 | |
| SK-NEP-1 | Ewings | 0.428 | 0.9 | >4 | 1.08 | 0.829 | |
| EW5 | Ewings | | >2.3! | 0.4! | 0.12 | | CR* |
| EW8 | Ewings | 0.579 | 1.5 | >4 | 0.86 | 0.243 | |
| TC-71 | Ewings | 0.246 | 1.2 | >4 | 0.88 | 0.579 | |
| CHLA258 | Ewings | | 4.2# | >4# | 0.29 | | PD2 |
| Rh28 | ALV Rhabdomyosarcoma | | >2.6# | 2.9# | 0.36 | | PD2 |
| Rh30R | ALV Rhabdomyosarcoma | | 1.8 | >4 | 0.49 | | PD2 |
| Rh41 | ALV Rhabdomyosarcoma | | 1.1 | >4 | 0.65 | 0.065 | |
| Rh18 | EMB Rhabdomyosarcoma | 0.44 | 1 | >4 | 0.74 | 0.353 | |
| BT-28 | Medulloblastoma | 0.121 | 1.2 | >4 | 1.18 | 0.529 | |
| BT-46 | Medulloblastoma | | 1.2 | >4 | 0.69 | | |
| BT-50 | Medulloblastoma | 0.474 | — | 1.4 | 0.94 | 0.436 | PD2 |
| BT-44 | Ependymoma | | 2.6# | >4# | 0.66 | | PD2 |
| GBM2 | Glioblastoma | | 1.4 | >4 | 0.73 | | |
| BT-39 | Glioblastoma | 0.377 | 1.1 | >4 | 0.87 | 0.631 | |
| D645 | Glioblastoma | | 2.2# | >4# | 0.58 | | PD2 |
| D456 | Glioblastoma | | 1.1 | >4 | 0.77 | | |
| NB-SD | Neuroblastoma | | >1.8\hat{ } | 3.7\hat{ } | 0.41 | | PD2 |
| NB-1771 | Neuroblastoma | 0.595 | 1.1 | >4 | 0.64 | 0.063 | |
| NB-1691 | Neuroblastoma | 0.025* | 0.9 | >4 | 1.34 | 0.075 | |
| NB-EBc1 | Neuroblastoma | | >2.0# | 1.4# | 0.8 | | PD2 |
| CHLA-79 | Neuroblastoma | | 2.1# | 3.9# | 0.69 | 0.123 | PD2 |
| NB-1643 | Neuroblastoma | | 1.8 | >4 | 0.53 | | PD2 |
| OS-1 | Osteosarcoma | | >1.3\hat{ } | 0.1\hat{ } | 0.16 | | |
| OS-2 | Osteosarcoma | | 2.3# | >4# | 0.5 | | PD2 |
| OS-17 | Osteosarcoma | | 2 | >4 | 0.55 | 0.089 | PD2 |
| OS-9 | Osteosarcoma | | >1.2\hat{ } | 0.4\hat{ } | 0.31 | | |
| OS-33 | Osteosarcoma | 0.587 | 0.9 | >4 | 0.79 | 0.529 | |
| OS-31 | Osteosarcoma | 0.461 | 1.3 | >4 | 0.88 | 0.274 | |
| ALL-2 | ALL B-precursor | 0.095* | 0.7 | >25 | — | | |
| ALL-3 | ALL B-precursor | 0.743 | 0.4 | >25 | — | | |
| ALL-4 | ALL B-precursor | 0.194 | 0.8 | >25 | — | | |
| ALL-7 | ALL B-precursor | 0.544 | 1 | >25 | — | | |
| ALL-8 | ALL T-cell | 0.408 | 1.5 | >25 | — | | |
| ALL-16 | ALL T-cell | 0.588 | 1 | >25 | — | | |
| ALL-17 | ALL B-precursor | | 3.1# | >25# | — | | PD2 |
| ALL-19 | ALL B-precursor | | 1.5 | >25 | — | | |

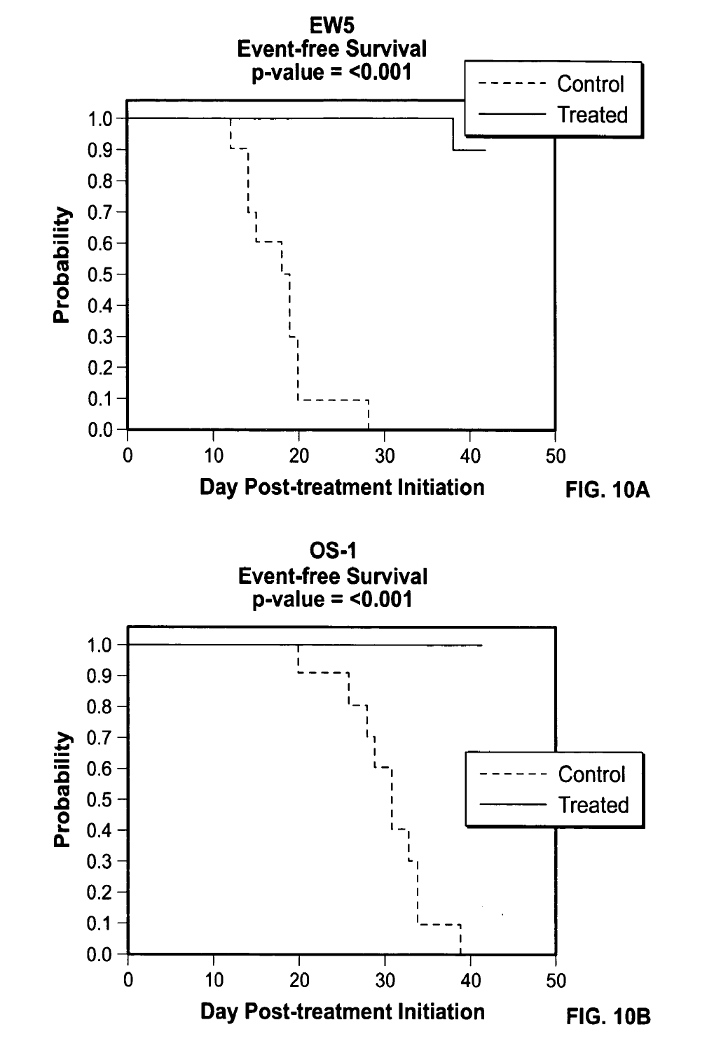

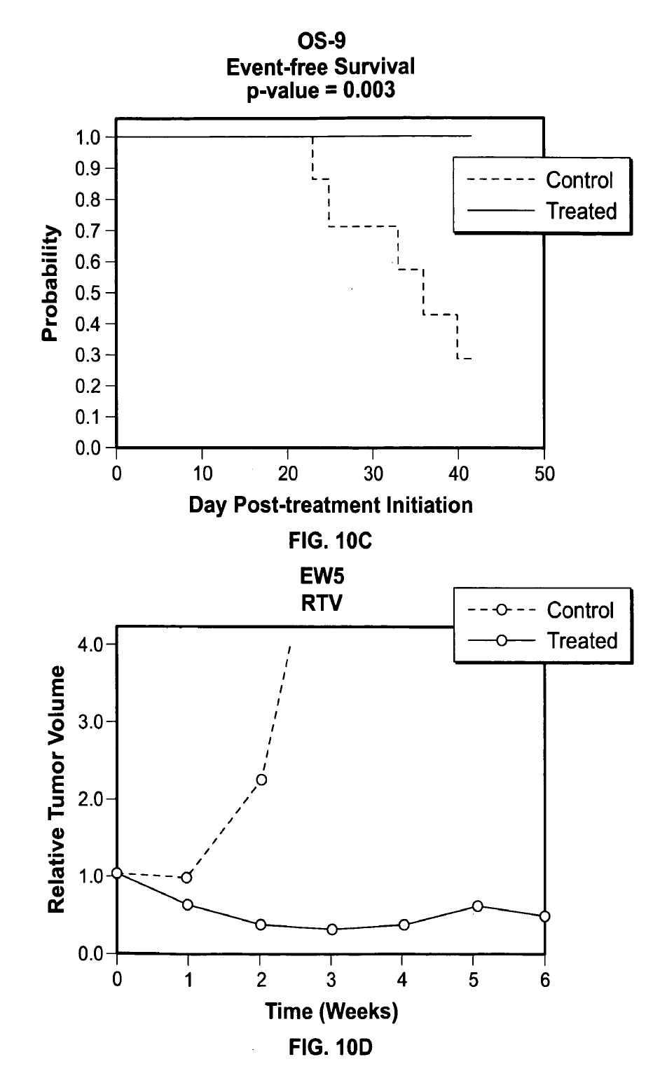

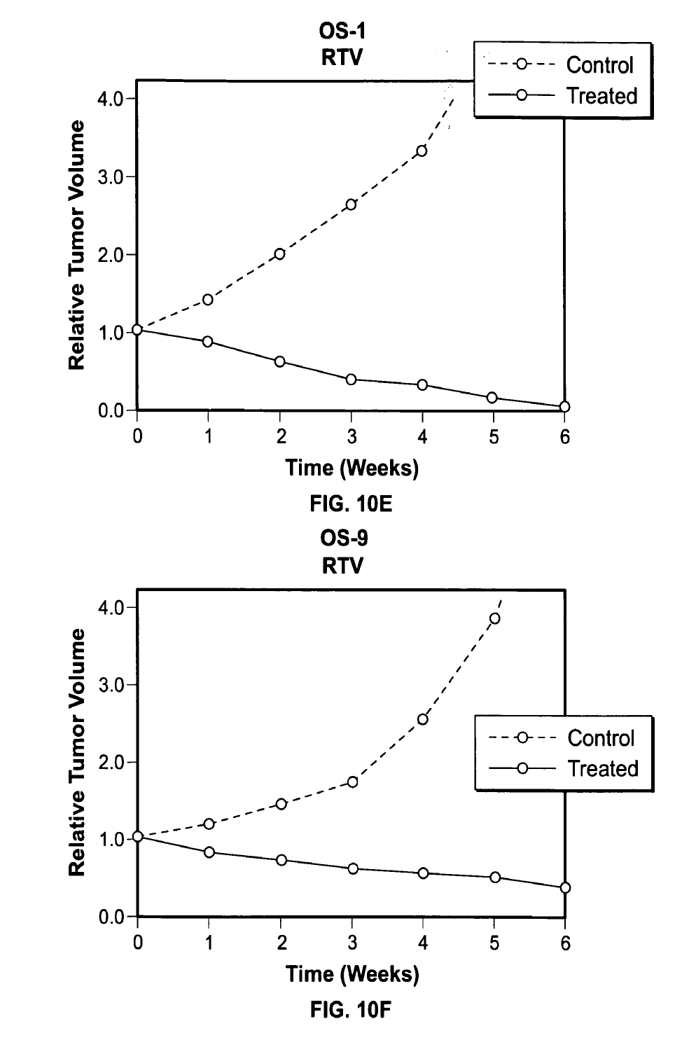

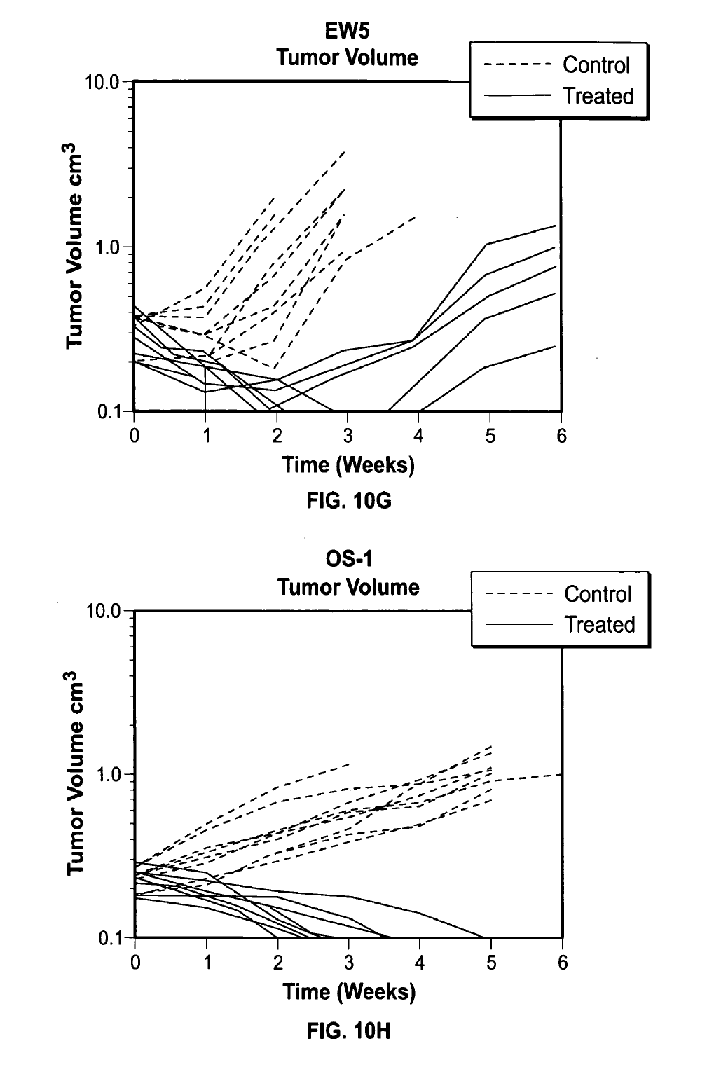

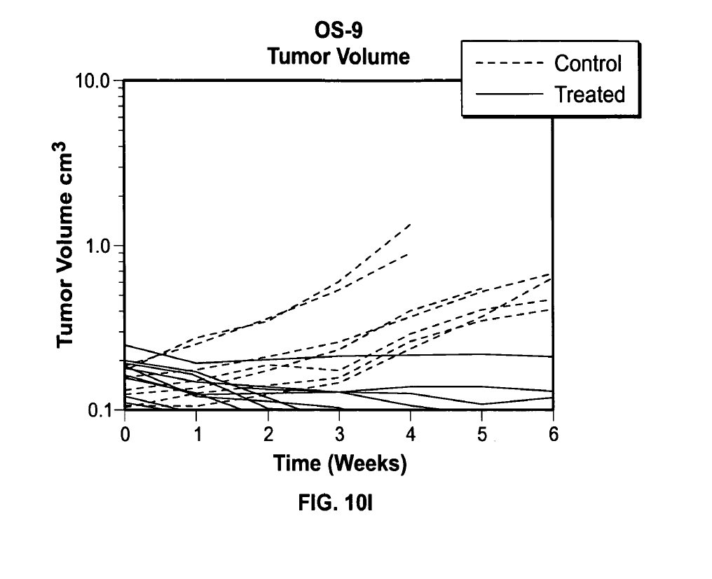

An * in the p-value columns indicates a statistically significant difference between treated and control groups. Notations in the EFS columns indicate xenografts that have either high (!), intermediate (#), or indeterminate (\hat{ }) activity. RTV is relative tumor volume.
See also FIG. 10 which sets forth a graphical analysis of the in vivo tumor volume of various cell types over time.
IGF-1R expression was also evaluated using Affymetrix U133 Plus2 Arrays.
Five xenografts with very low IGF1R expression did not respond (i.e., PD1) to the antibody. For example, OS-33 had very low expression and was one of two osteosarcoma xenografts that showed no response to the antibody. The 3 xenografts with CR or MCR responses had high IGF1R expression. Some xenografts with moderate to high IGF1R expression did not respond to the antibody (e.g., Rh41).
The maximal growth inhibition achieved in the in vitro assays was 30% and was observed for the T-cell ALL line, MOLT-4. The median growth inhibition for the in vitro panel, at the highest concentration tested, was 5%.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. A method for treating or preventing a medical condition, in a mammalian subject, selected from the group consisting of head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphoblastic leukemia of the T-cell lineage, acute lymphoblastic leukemia of the B-cell precursor lineage, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, cental nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumor and liver cancer comprising administering a therapeutically effective amount of one or more IGF1R inhibitors or pharmaceutical compositions thereof to the subject.

2. The method of claim 1 wherein the IGF1R inhibitor is selected from the group consisting of 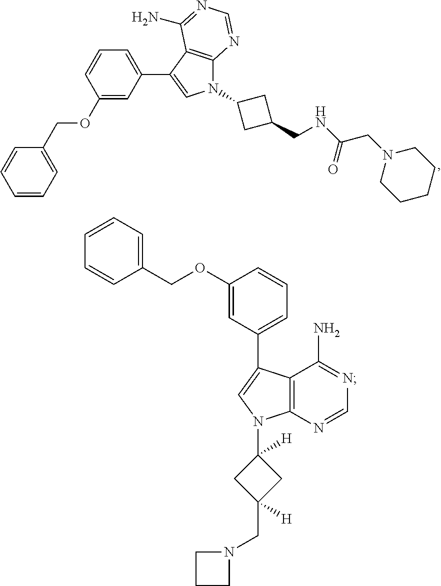
and an isolated antibody that binds specifically to human IGF1R or an antigen-binding fragment thereof.

3. The method of claim 2 wherein the inhibitor is an antibody or antigen-binding fragment thereof comprising CDR-L1, CDR-L2 and CDR-L3 from a light chain immunoglobulin comprising the amino acid sequence of SEQ ID NO: 2, 4, 6, or 8; and/or CDR-H1, CDR-H2 and CDR-H3 from a heavy chain immunoglobulin comprising the amino acid sequence of SEQ ID NO: 10 or 12.

4. The method of claim 2 wherein the inhibitor is an antibody or antigen-binding fragment thereof comprising CDR-L1, CDR-L2 and CDR-L3 from a light chain immunoglobulin comprising the amino acid sequence of SEQ ID NO: 25, 26, 27 or 28; and/or CDR-H1, CDR-H2 and CDR-H3 from a heavy chain immunoglobulin comprising the amino acid sequence of SEQ ID NO: 29, 30, 31, 32 or 33.

5. The method of claim 2 wherein the inhibitor is an antibody wherein said antibody comprises:
(a) a light chain variable region comprising amino acids 20-128 of SEQ ID NO: 2 and a heavy chain variable region comprising amino acids 20-137 of SEQ ID NO: 10 or 12;
(b) a light chain variable region comprising amino acids 20-128 of SEQ ID NO: 4 and a heavy chain variable region comprising amino acids 20-137 of SEQ ID NO: 10 or 12;
(c) a light chain variable region comprising amino acids 20-128 of SEQ ID NO: 6 and a heavy chain variable region comprising amino acids 20-137 of SEQ ID NO: 10 or 12; or
(d) a light chain variable region comprising amino acids 20-128 of SEQ ID NO: 8 and a heavy chain variable region comprising amino acids 20-137 of SEQ ID NO: 10 or 12.

6. The method of claim 1 wherein the IGF1R inhibitor is administered in association with one or more further chemotherapeutic agents or a pharmaceutical composition thereof.

7. The method of claim 6 wherein the further chemotherapeutic agent is an anti-cancer agent, an anti-emetic agent, an anti-anemic agent or an anti-mucositis agent.

8. The method of claim 6 wherein the further chemotherapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 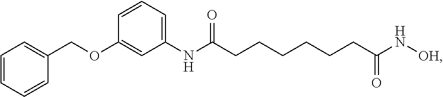
CG-781, CG-1521 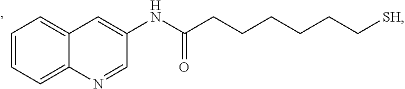
SB-556629, chlamydocin, JNJ-16241199, 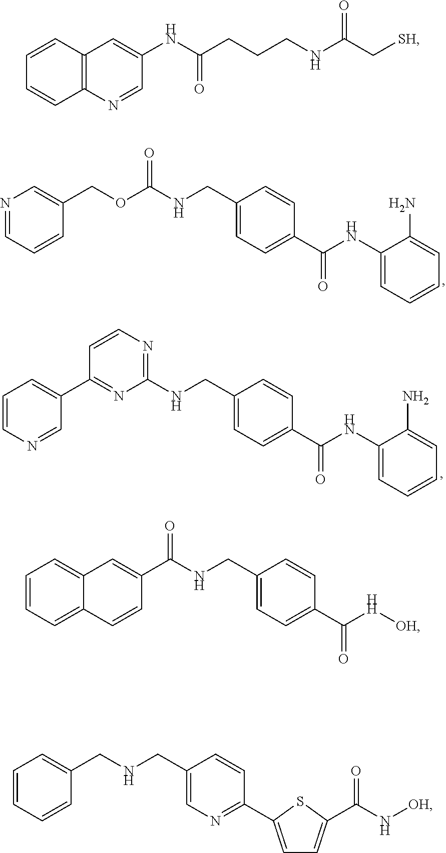
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 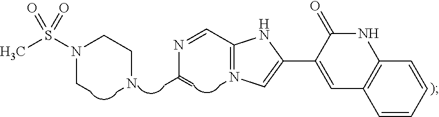
3-[5-(methylsulfonylpiperadinemethyl)-indolyl]-quinolone, vatalanib, AG-013736, AVE-0005, the acetate salt of [D-Ser(Bu t) 6 ,Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 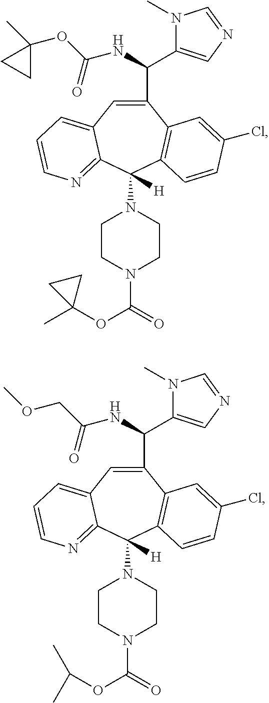
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, satraplatin pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa

9. The method of claim 6 wherein the IGF1R inhibitor and the further anti-cancer therapeutic agent are administered simultaneously.

10. The method of claim 6 wherein the IGF1R inhibitor and the further anti-cancer therapeutic agent are administered non-simultaneously.

11. The method of claim 2 wherein the antibody comprises an IgG constant region.

12. The method of claim 1 wherein the subject is a human.

13. The method of claim 12 wherein the subject is a child.

14. The method of claim 1 wherein the IGF1R inhibitor is administered in association with an anti-cancer therapeutic procedure.

15. The method of claim 14 wherein the anti-cancer therapeutic procedure is surgical tumorectomy and/or anti-cancer radiation treatment.
